(19) United States
(12) Patent Application Publication
(10) Pub. No.: US 2015/0174123 A1
(43) Pub. Date: Jun. 25, 2015

(54) OXABICYCLOHEPTANES AND OXABICYCLOHEPTENES FOR THE TREATMENT OF REPERFUSION INJURY

(71) Applicant: John S. KOVACH, East Setauket, NY (US)

(72) Inventor: John S. Kovach, East Setauket, NY (US)

(73) Assignee: Lixte Biotechnology, Inc., East Setauket, NY (US)

(21) Appl. No.: 14/408,208

(22) PCT Filed: Jun. 28, 2013
(86) PCT No.: PCT/US13/48697

§ 371 Date: Dec. 15, 2014

Related U.S. Application Data
(60) Provisional application No. 61/666,535, filed on Jun. 29, 2012.
(60) Provisional application No. 61/782,894, filed on Mar. 14, 2013.

(51) Int. Cl. A61K 31/496

(52) U.S. Cl. CPC A61K 31/496

(57) ABSTRACT

A method of reducing reperfusion injury in mammalian tissue comprising contacting the tissue with a protein phosphatase 2A (PP2A) inhibitor having the structure:

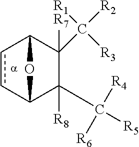

Throughout this application various publications are referenced. The disclosures of these documents in their entireties are hereby incorporated by reference into this application in order to more fully describe the state of the art to which this invention pertains.

### BACKGROUND OF THE INVENTION

Reperfusion is a re-establishment of blood flow and re-oxygentaion of an affected area following an ischemic event and is critical to limit irreversible damage. However, the absence of oxygen and nutrients from the blood creates a condition in which reperfusion injury may occur. The restoration of blood flow after an ischemic event results in inflammation and oxidative damage. Upon restoration of blood flow, white blood cells release inflammatory factors such as interleukins as well as free radicals. The restored blood flow reintroduces oxygen within cells that damages cellular proteins, DNA, and the plasma membrane.

As acute myocardial infarction (MI) remains the leading cause of death worldwide, the possibility that a pharmacologic intervention applied promptly but after the onset of the heart attack would minimize damage to heart tissue caused by reperfusion and therefore be expected to save many lives and reduce the number of individuals with heart failure following excessive cardiac muscle damage after an MI (Yellon and Hausenloy, 2005; Longacre et al, 2011). It has been suggested that a lack of commercial interest in a developing a drug that would likely be used only once in an individual has limited progress in this field (Cohen and Downey, 2011).

At present, the only established intervention that consistently reduces the size of myocardial infarcts in humans is by improving coronary artery flow as soon as possible after an MI either by drugs, which dissolve fresh clots and/or cardiac catheterization with balloon angioplasty with or without placement of a fixed conduit, a stent. These methods of improving coronary artery blood flow (reperfusion) have improved patient care and decreased hospital mortality. However, delay in initiating reperfusion because of travel time to a cardiac center is a serious limitation to applying these treatments to patients with acute cardiac injury. It has also been discovered the reperfusion treatment in and of itself may cause myocardial cell death, a phenomenon called reperfusion injury. Reducing injury caused by reperfusion by pharmacologic means should improve the success of current interventions for acute heart attacks. A drug minimizing tissue damage that could be administered at the time of a MI by emergency personnel prior to arrival at a cardiac center could be a major advance in the care of heart attack victims. Acute injury due to oxygen deprivation leading to myocardial damage is also a significant problem in heart surgery. The incidence of infarction after coronary artery bypass graft surgery has been estimated to be as high as 19% with attendant cardiac morbidity (Longacre et al, 2011).

### SUMMARY OF THE INVENTION

A method of reducing reperfusion injury in mammalian tissue comprising contacting the tissue with a protein phosphatase 2A (PP2A) inhibitor having the structure:

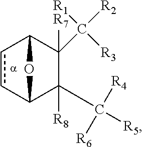

wherein
bond α is present or absent;
R₁ and R₂ is each independently H, O⁻ or OR₉,
   where R₉ is H, alkyl, alkenyl, alkynyl or aryl,
   or R₁ and R₂ together are ═O;
R₃ and R₄ are each different, and each is OH, O⁻, OR₉, OR₁₀, O(CH₂)₁₋₆R₉, SH, S⁻, SR₉,

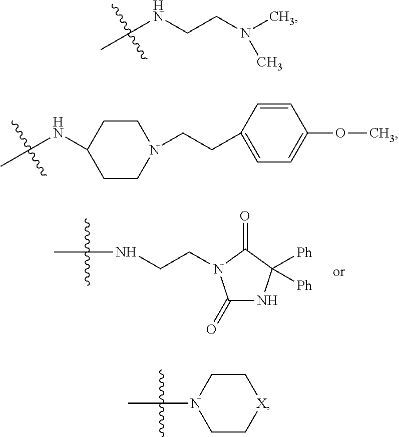

where X is O, S, NR₁₀, or N⁺R₁₀R₁₀,
   where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O,

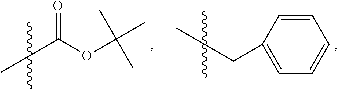

—CH₂CN, —CH₂CO₂R₁₁, —CH₂COR₁₁, —NHR₁₁ or —NH⁺(R₁₁)₂,
   wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
   where R₁₂ is H, alkyl, alkenyl, alkynyl or aryl; and
each occurrence of alkyl, alkenyl, or alkynyl is branched or unbranched, unsubstituted or substituted,
or a salt, enantiomer or zwitterion of the compound.

A method of reducing tissue damage associated with reperfusion injury in the heart of a subject following a myocardial infarction comprising administering to the subject a therapeutically effective amount of a protein phosphatase 2A (PP2A) inhibitor having the structure:

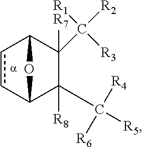

wherein
bond α is present or absent;
R₁ and R₂ is each independently H, O⁻ or OR₉,
   where R₉ is H, alkyl, alkenyl, alkynyl or aryl,
   or R₁ and R₂ together are ═O;
R₃ and R₄ are each different, and each is OH, O⁻, OR₉, OR₁₀, O(CH₂)₁₋₆R₉, SH, S⁻, SR₉,

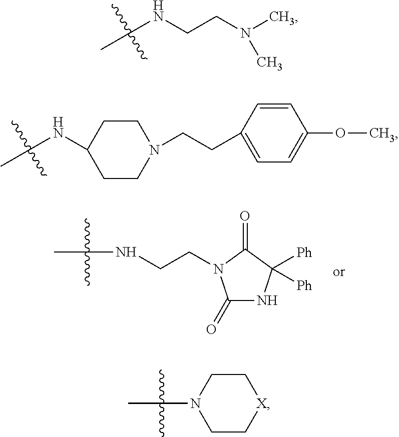

where X is O, S, NR₁₀, or N⁺R₁₀R₁₀,
   where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O,

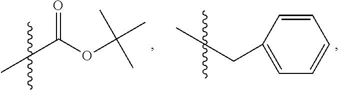

—CH₂CN, —CH₂CO₂R₁₁, —CH₂COR₁₁, —NHR₁₁ or —NH⁺(R₁₁)₂,
   wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
   where R₁₂ is H, alkyl, alkenyl, alkynyl or aryl; and
each occurrence of alkyl, alkenyl, or alkynyl is branched or unbranched, unsubstituted or substituted,
or a salt, enantiomer or zwitterion of the compound.

A method of reducing vascular leakage associated with reperfusion injury in a subject suffering from sepsis comprising administering to the subject a therapeutically effective amount of a protein phosphatase 2A (PP2A) inhibitor having the structure:

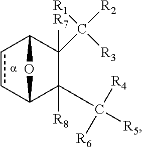

wherein
bond α is present or absent;
R₁ and R₂ is each independently H, O⁻ or OR₉,
   where R₉ is H, alkyl, alkenyl, alkynyl or aryl, or R₁ and R₂ together are ═O;
R₃ and R₄ are each different, and each is OH, O⁻, OR₉, OR₁₀, O(CH₂)₁₋₆R₉, SH, S⁻, SR₉,

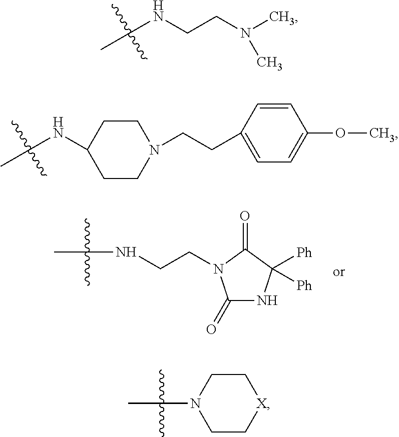

where X is O, S, NR₁₀, or N⁺R₁₀R₁₀,
   where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O,

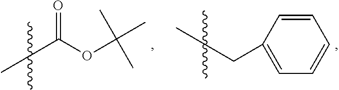

—CH₂CN, —CH₂CO₂R₁₁, —CH₂COR₁₁, —NHR₁₁ or —NH⁺(R₁₁)₂,
   wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
   where R₁₂ is H, alkyl, alkenyl, alkynyl or aryl; and
each occurrence of alkyl, alkenyl, or alkynyl is branched or unbranched, unsubstituted or substituted,
or a salt, enantiomer or zwitterion of the compound.

### BRIEF DESCRIPTION OF THE FIGURES

FIG. 1. Photographs of in-bred “control” rats 1-10 implanted with a pump containing sodium chloride prior to raising a skin flap. Photographs were taken 7 days after creation of the skin flap. (A) control rat 1; (B) control rat 2; (C) control rat 3; (D) control rat 4; (E) control rat 5; (F) control rat 6; (G) control rat 7; (H) control rat 8; (I) control rat 9; and (J) control rat 10.

FIG. 2. Photographs of in-bred “treatment” rats 1-10 implanted with a pump containing 0.55 mg of LB-100 prior to raising a skin flap. The pump was set to deliver 0.5 μl/hour+/−10% so as to administer about 12 μl/day for 8 days, four days prior to raising the graft and for the first four days after creation of the graft. Photographs were taken 7 days after creation of the skin flap. (A) treatment rat 1; (B) treatment rat 2; (C) treatment rat 3; (D) treatment rat 4; (E) treatment rat 5; (F) treatment rat 6; (G) treatment rat 7; (H) treatment rat 8; (I) treatment rat 9; and (J) treatment rat 10.

### DETAILED DESCRIPTION OF TEE INVENTION

A method of reducing reperfusion injury in mammalian tissue comprising contacting the tissue with a protein phosphatase 2A (PP2A) inhibitor having the structure:

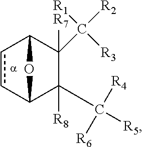

wherein
bond α is present or absent;
R₁ and R₂ is each independently H, O⁻ or OR₉,
   where R₉ is H, alkyl, alkenyl, alkynyl or aryl,
   or R₁ and R₂ together are ═O;
R₃ and R₄ are each different, and each is OH, O⁻, OR₉, OR₁₀, O(CH₂)₁₋₆R₉, SH, S⁻, SR₉,

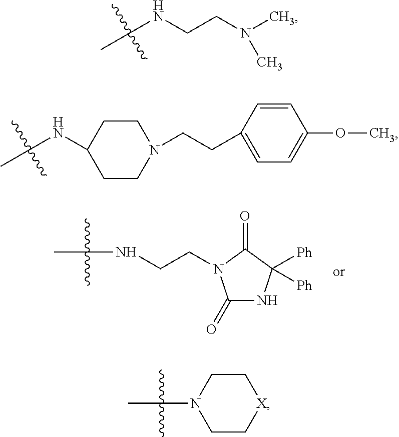

where X is O, S, NR₁₀, or N⁺R₁₀R₁₀,
   where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O,

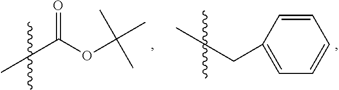

—CH₂CN, —CH₂CO₂R₁₁, —CH₂COR₁₁, —NHR₁₁ or —NH⁺(R₁₁)₂,
   wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
   where R₁₂ is H, alkyl, alkenyl, alkynyl or aryl; and
each occurrence of alkyl, alkenyl, or alkynyl is branched or unbranched, unsubstituted or substituted,
or a salt, enantiomer or zwitterion of the compound.

In some embodiments, the method wherein the reduction of reperfusion injury comprises increased phosphorylation of Akt in the mammalian tissue that has suffered an ischemia.

In some embodiments, the method wherein the reduction of reperfusion injury comprises increased activation of Akt in the mammalian tissue that has suffered an ischemia.

In some embodiments, the method wherein the reduction of reperfusion injury comprises increased phosphorylation of BAD, mdm2, eNOS and/or GSK-3β in the mammalian tissue that has suffered an ischemia.

In some embodiments, the method wherein the ischemia is caused by a myocardial infarction, stroke or sepsis.

In some embodiments, the method wherein the tissue is myocardial tissue, brain tissue or endothelial tissue.

In some embodiments, the method wherein endothelial dysfunction is reduced.

In some embodiments, the method wherein the tissue is myocardial tissue, brain tissue or endothelial tissue.

A method of reducing tissue damage associated with reperfusion injury in the heart of a subject following a myocardial infarction comprising administering to the subject a therapeutically effective amount of a protein phosphatase 2A (PP2A) inhibitor having the structure:

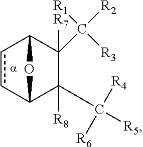

wherein
bond α is present or absent;
R₁ and R₂ is each independently H, O⁻ or OR₉,
   where R₉ is H, alkyl, alkenyl, alkynyl or aryl,
   or R₁ and R₂ together are ═O;
R₃ and R₄ are each different, and each is OH, O⁻, OR₉, OR₁₀, O(CH₂)₁₋₆R₉, SH, S⁻, SR₉,

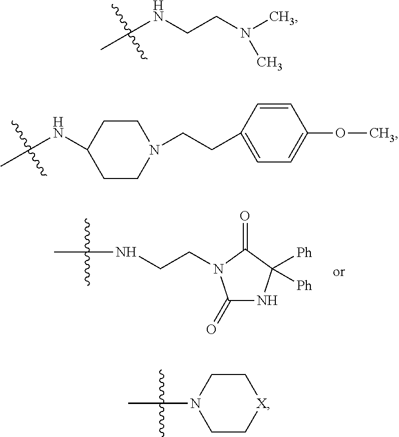

where X is O, S, NR₁₀, or N⁺R₁₀R₁₀,
   where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O,

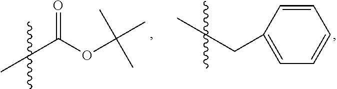

—CH₂CN, —CH₂CO₂R₁₁, —CH₂COR₁₁, —NHR₁₁ or —NH⁺(R₁₁)₂,
   wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
   where R₁₂ is H, alkyl, alkenyl, alkynyl or aryl; and
each occurrence of alkyl, alkenyl, or alkynyl is branched or unbranched, unsubstituted or substituted,
or a salt, enantiomer or zwitterion of the compound.

A method of reducing vascular leakage associated with reperfusion injury in a subject suffering from sepsis comprising administering to the subject a therapeutically effective amount of a protein phosphatase 2A (PP2A) inhibitor having the structure:

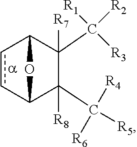

wherein
bond α is present or absent;
R₁ and R₂ is each independently H, O⁻ or OR₉,
   where R₉ is H, alkyl, alkenyl, alkynyl or aryl,
   or R₁ and R₂ together are ═O;
R₃ and R₄ are each different, and each is OH, O⁻, OR₉, OR₁₀, O(CH₂)₁₋₆R₉, SH, S⁻, SR₉,

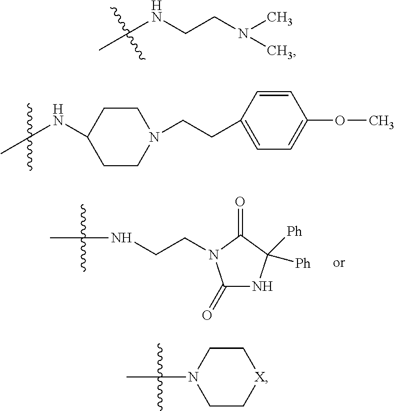

where X is O, S, NR₁₀, or N⁺R₁₀R₁₀,
   where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O,

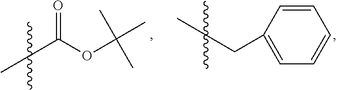

—CH₂CN, —CH₂CO₂R₁₁, —CH₂COR₁₁, —NHR₁₁ or —NH⁺(R₁₁)₂,
   wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
   where R₁₂ is H, alkyl, alkenyl, alkynyl or aryl; and
each occurrence of alkyl, alkenyl, or alkynyl is branched or unbranched, unsubstituted or substituted,
or a salt, enantiomer or zwitterion of the compound.

In some embodiments, wherein the reperfusion injury is caused by ischemia that is caused by septic shock.

In one embodiment, the protein phosphatase 2A inhibitor has the structure

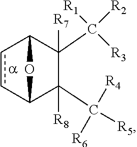

wherein
bond α is present or absent;
R₁ and R₂ is each independently H, O⁻ or OR₉,
   where R₉ is H, alkyl, alkenyl, alkynyl or aryl,
   or R₁ and R₂ together are ═O;
R₃ and R₄ are each different, and each is OH, O⁻, OR₉, OR₁₀, O(CH₂)₁₋₆R₉, SH, S⁻, SR₉,

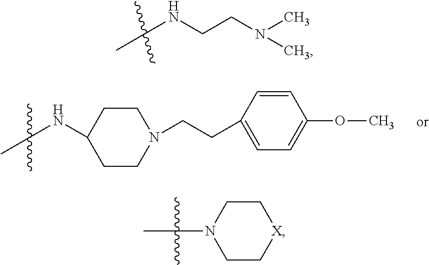

where X is O, S, NR₁₀, or N⁺R₁₀R₁₀,
   where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O,

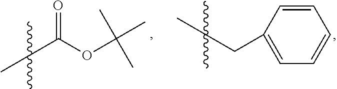

—CH₂CN, —CH₂CO₂R₁₁, —CH₂COR₁₁, —NHR₁₁ or —NH⁺(R₁₁)₂,
   wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂, where R₁₂ is H, alkyl, alkenyl, alkynyl or aryl; and
   or a salt, enantiomer or zwitterion of the compound.

In one embodiment, the protein phosphatase 2A inhibitor has the structure

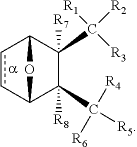

In one embodiment, the protein phosphatase 2A inhibitor has the structure

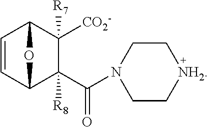

In one embodiment, the protein phosphatase 2A inhibitor has the structure

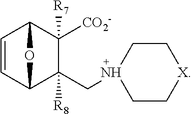

In one embodiment, bond α is present. In another embodiment, bond α is absent.

In one embodiment, R₁ and R₂ together are ═O;

R₃ is O⁻ or OR₉, where R₉ is H, methyl, ethyl or phenyl;

R₄ is

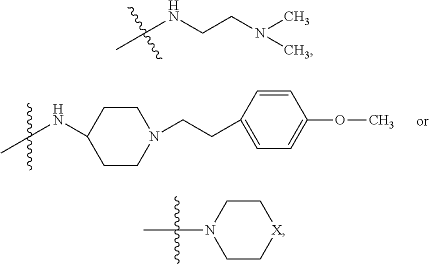

where X is O, S, NR₁₀, or N⁺R₁₀R₁₀,
   where each R₁₀ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro,

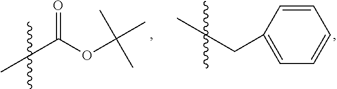

—CH₂CN, —CH₂CO₂R₁₁, —CH₂COR₁₁, —NHR₁₁ or —NH⁺(R₁₁)₂,
   where R₁₁ is alkyl, alkenyl or alkynyl, each of which is substituted or unsubstituted, or H;

R₅ and R₆ taken together are ═O; and

R₇ and R₈ is each independently H, F, Cl, Br, SO₂Ph, CO₂CH₃, or SR₁₂, where R₁₂ is a substituted or unsubstituted alkyl, alkenyl or alkynyl.

In one embodiment, R₃ is O⁻.

In another embodiment, R₄ is

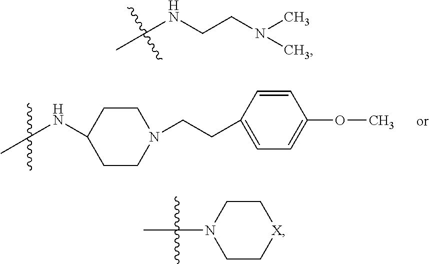

where X is O, NR₁₀, NR₁₀, N⁺R₁₀R₁₀
   where each R₁₀ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro when R₁ and R₂ are ═O,

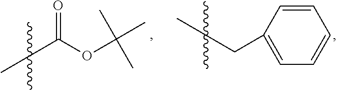

—CH₂CN, —CH₂CO₂R₁₁, —CH₂COR₁₁, —NHR₁₁ or —NH⁺(R₁₁)₂,
   where R₁₁ is H or alkyl.

In one embodiment, the protein phosphatase inhibitor 2A has the structure

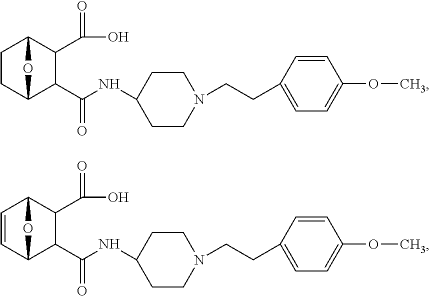

In one embodiment, R₄ is

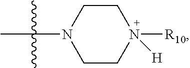

where R₁₀ is H, alkyl, substituted C₂-C₁₂ alkyl, alkenyl, substituted C₄-C₁₂ alkenyl, alkynyl, substituted alkynyl, aryl, substituted aryl where the substituent is other than chloro when R₁ and R₂ are ═O,

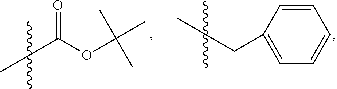

—CH₂CN, —CH₂CO₂R₁₁, —CH₂COR₁₁, —NHR₁₁ or —NH⁺(R₁)₂, where R₁₁ is H or alkyl.

In one embodiment, R₄ is

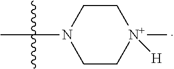 [SMILES: *N1CC[NH+](C)CC1]

In one embodiment, R₄ is

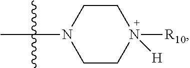

where R₁₀ is

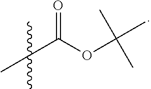 [SMILES: *C(=O)OC(C)(C)C]

In one embodiment, R₄ is

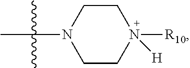

where R₁₀ is

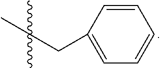 [SMILES: *Cc1ccccc1]

In one embodiment, R₄ is

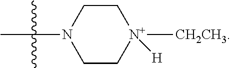 [SMILES: *N1CC[NH+](CC)CC1]

In one embodiment, R₄ is

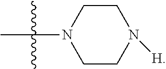

In one embodiment, R₅ and R₆ together are ═O. In another embodiment, R₇ and R₈ are each H.

In one embodiment,

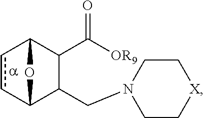

wherein bond α is present or absent;

R₉ is present or absent and when present is H, C₁-C₁₀ alkyl, C₂-C₁₀ alkenyl or phenyl; and X is O, S, NR₁₀ or N⁺R₁₀R₁₀,
where each R₁₀ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro,

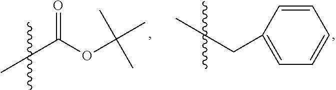

—CH₂CO₂R₁₁, —CH₂COR₁₁, —CH₂CN, or —CH₂CH₂R₁₆, where R₁₁ is H or alkyl, and where R₁₆ is any substitutent that is a precursor to an aziridinyl intermediate,
   or a salt, zwitterion or enantiomer of the compound.

In one embodiment, the protein phosphatase 2A inhibitor has the structure

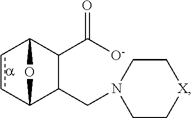

wherein,

bond α is present or absent;

X is O, S, NR₁₀ or N⁺R₁₀R₁₀,
where each R₁₀ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro,

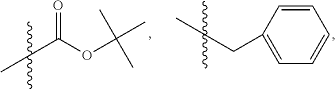

—CH₂CO₂R₁₁, —CH₂COR₁₁, —CH₂CN, or —CH₂CH₂R₁₆, where R₁₁ is H or alkyl, and where R₁₆ is any substitutent that is a aziridinyl intermediate,
   or a salt, zwitterion or enantiomer of a compound.

In one embodiment,

X is O or NH⁺R₁₀,
where R₁₀ is H, alkyl, substituted C₂-C₁₂ alkyl, alkenyl, substituted C₄-C₁₂ alkenyl, alkynyl, substituted alkynyl, aryl, substituted aryl where the substituent is other than chloro,

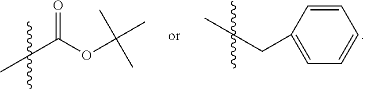

In one embodiment, X is —CH₂CH₂R₁₆, where R₁₆ is any substitutent that is a precursor to an aziridinyl intermediate.

In one embodiment, X is O.

In another embodiment, X is NH⁺R₁₀,
where R₁₀H, alkyl, substituted C₂-C₁₂ alkyl, alkenyl, substituted C₄-C₁₂ alkenyl, alkynyl, substituted alkynyl, aryl, substituted aryl where the substituent is other than chloro,

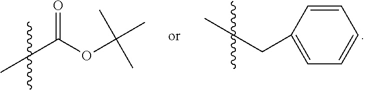

In one embodiment, R₁₀ is methyl. In another embodiment, R₁₀ is

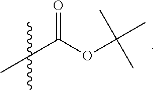 [SMILES: *C(=O)OC(C)(C)C]

In one embodiment, R₁₀ is

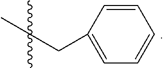 [SMILES: *Cc1ccccc1]

In one embodiment, R₁₀ is ethyl. In another embodiment, R₁₀ is absent.

In one embodiment, the protein phosphatase 2A inhibitor has the structure

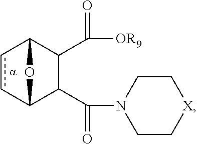

wherein

bond α is present or absent;

R₉ is present or absent and when present is H, alkyl, alkenyl, alkynyl or phenyl; and

X is O, NR₁₀, or N⁺R₁₀R₁₀,
where each R₁₀ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro,

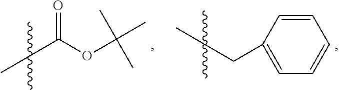

—CH₂CN, —CH₂CO₂R₁₂, or —CH₂COR₁₂,
   where R₁₂ is H or alkyl,
      or a salt, zwitterion, or enantiomer of the compound.

In one embodiment, the protein phosphatase 2A inhibitor has the structure

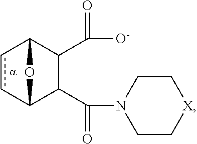

wherein
bond α is present or absent;
X is O or NH⁺R₁₀,
   where R₁₀ is H, alkyl, substituted C₂-C₁₂ alkyl, alkenyl, substituted C₄-C₁₂ alkenyl, alkynyl, substituted alkynyl, aryl, substituted aryl where the substituent is other than chloro,

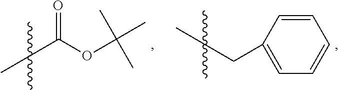

—CH₂CN, —CH₂CO₂R₁₂, or —CH₂COR₁₂, where R₁₂ is H or alkyl.

In one embodiment, bond α is present. In another embodiment, bond α is absent.

In one embodiment, the protein phosphatase 2A inhibitor has the structure

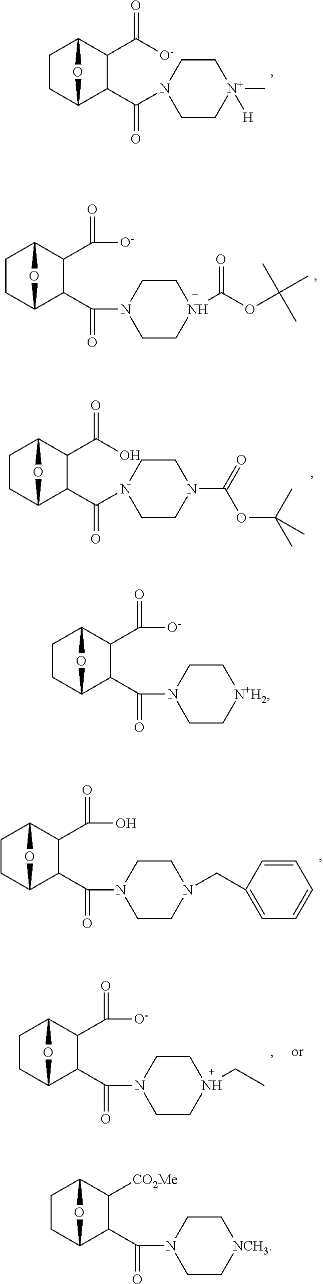

In one embodiment, the protein phosphatase 2A inhibitor has the structure

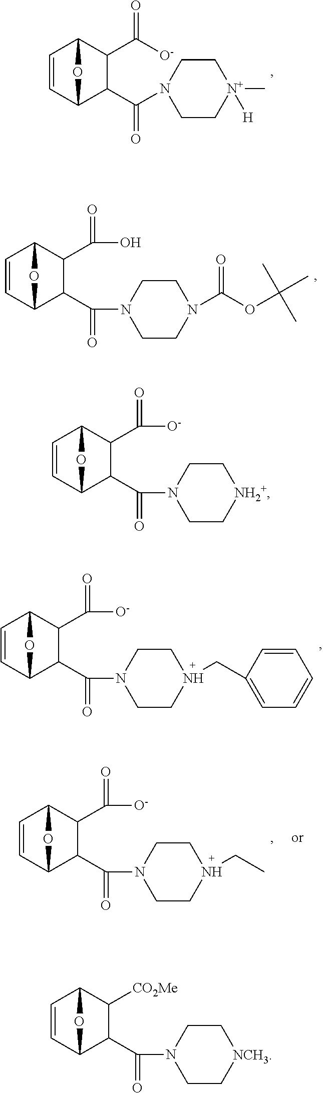

In one embodiment, the protein phosphatase 2A inhibitor has the structure

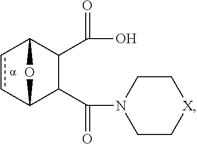

wherein

bond α is present or absent;

X is NH⁺R₁₀,
where R₁₀ is present or absent and when present R₁₀ is alkyl, substituted C2-C12 alkyl, alkenyl, substituted C4-C12 alkenyl,

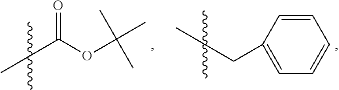

—CH₂CN, —CH₂CO₂R₁₂, or —CH₂COR₁₂, where R₁₂ is H or alkyl.

In one embodiment of the method, the protein phosphatase 2A inhibitor has the structure

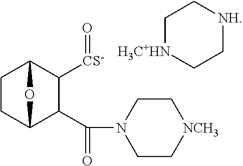

In one embodiment of the method, the protein phosphatase 2A inhibitor has the structure

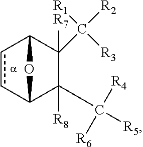

wherein
bond α is present or absent;
R₁ and R₂ is each independently H, O⁻ or OR₉,
   where R₉ is H, alkyl, substituted alkyl, alkenyl, alkynyl or aryl,
or R₁ and R₂ together are ═O;
R₃ and R₄ are each different, and each is O(CH₂)₁₋₆R₉ or OR₉,
or

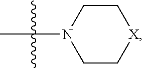

where X is O, S, NR₁₀, or N⁺R₁₀R₁₀,
   where each R₁₀ is independently H, alkyl, hydroxyalkyl, C₂-C₁₂ alkyl, alkenyl, C₄-C₁₂ alkenyl, alkynyl, aryl, substituted aryl where the substituent is other than chloro when R₁ and R₂ are ═O,

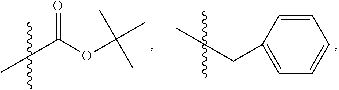

—CH₂CN, —CH₂CO₂R₁₁, —CH₂COR₁₁, —NHR₁₁ or —NH⁺(R₁₁)₂,
   where each R₁₁ is independently alkyl, alkenyl or alkynyl, each of which is substituted or unsubstituted, or H;
or R₃ and R₄ are each different and each is OH or

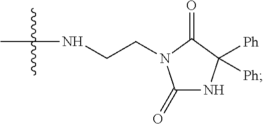 [SMILES: *NCCN1C(=O)NC(c2ccccc2)(c2ccccc2)C1=O]

R₅ and R₆ is each independently H, OH, or R₅ and R₆ taken together are ═O;
R₇ and R₈ is each independently H, F, Cl, Br, SO₂Ph, CO₂CH₃, or SR₁₂,
   where R₁₂ is H, aryl or a substituted or unsubstituted alkyl, alkenyl or alkynyl; and
each occurrence of alkyl, alkenyl, or alkynyl is branched or unbranched, unsubstituted or substituted,
   or a salt, enantiomer or zwitterion of the compound.

In one embodiment of the method, the protein phosphatase 2A inhibitor has the structure

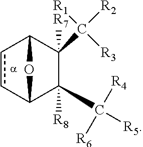

In one embodiment of the method, the bond α is present.

In one embodiment of the method, the bond α is absent.

In one embodiment of the method,

R₃ is OR₉ or O(CH₂)₁₋₆R₉,
where R₉ is aryl, substituted ethyl or substituted phenyl,
wherein the substituent is in the para position of the phenyl;

R₄ is

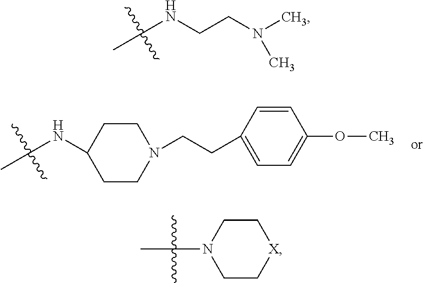

where X is O, S, NR₁₀, or N⁺R₁₀R₁₀,
where each R₁₀ is independently H, alkyl, hydroxyalkyl, substituted C₂-C₁₂ alkyl, alkenyl, substituted C₄-C₁₂ alkenyl, alkynyl, substituted alkynyl, aryl, substituted aryl where the substituent is other than chloro,

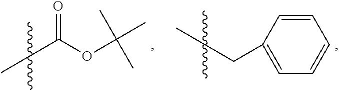

—CH₂CN, —CH₂CO₂R₁₁, —CH₂COR₁₁, —NHR₁₁ or —NH⁺(R₁₁)₂,
   where R₁₁ is alkyl, alkenyl or alkynyl, each of which is substituted or unsubstituted, or H;
      or where R₃ is OH and R₄ is

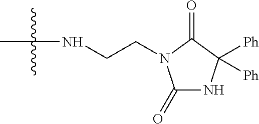 [SMILES: *NCCN1C(=O)NC(c2ccccc2)(c2ccccc2)C1=O]

In one embodiment of the method,

R₄ is

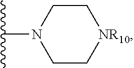

where R₁₀ is alkyl or hydroxylalkyl

### or R₄ is

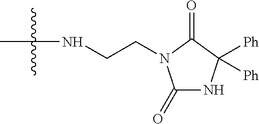 [SMILES: *NCCN1C(=O)NC(c2ccccc2)(c2ccccc2)C1=O]

when R₃ is OH.

In one embodiment of the method,
R₁ and R₂ together are ═O;
R₃ is OR₉ or O(CH₂)₁₋₂R₉,
   where R₉ is aryl, substituted ethyl, or substituted phenyl, wherein the substituent is in the para position of the phenyl;
or R₃ is OH and R₄ is

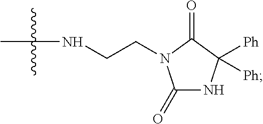 [SMILES: *NCCN1C(=O)NC(c2ccccc2)(c2ccccc2)C1=O]

R₄ is

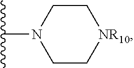

where R₁₀ is alkyl or hydroxyl alkyl;
R₅ and R₆ together are ═O; and
R₇ and R₈ are each independently H.

In one embodiment of the method,
R₁ and R₂ together are ═O;
R₃ is OH, O(CH₂)R₉, or OR₉,
   where R₉ is phenyl or CH₂CCl₃,

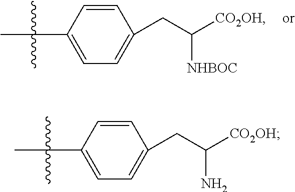

R₄ is

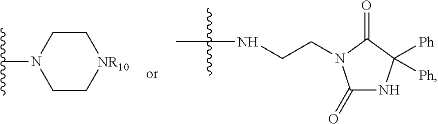

where R₁₀ is CH₃ or CH₃CH₂OH;
R₅ and R₆ together are ═O; and
R₇ and R₈ are each independently H.

In one embodiment of the method,
R₃ is OR₉
   where R₉ is (CH₂)₁₋₆(CHNHBOC)CO₂H, (CH₂)₁₋₆(CHNH₂)CO₂H, or (CH₂)₁₋₆CCl₃.

In one embodiment of the method,
R₉ is CH₂(CHNHBOC)CO₂H, CH₂(CHNH₂)CO₂H, or CH₂CCl₃.

In one embodiment of the method,
R₃ is O(CH₂)₁₋₆R₉ or O(CH₂)R₉,
   where R₉ is phenyl.

In one embodiment of the method,
R₃ is O(CH₂)R₉
   where R₉ is phenyl.

In one embodiment of the method,
R₃ is OH and R₄ is

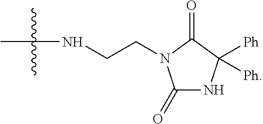 [SMILES: *NCCN1C(=O)NC(c2ccccc2)(c2ccccc2)C1=O]

In one embodiment of the method,
R₄ is

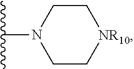

wherein R₁₀ is alkyl or hydroxyalkyl.

In one embodiment of the method, R₁₁ is —CH₂CH₂OH or —CH₃.

In one embodiment of the method, the protein phosphatase 2A inhibitor has the structure

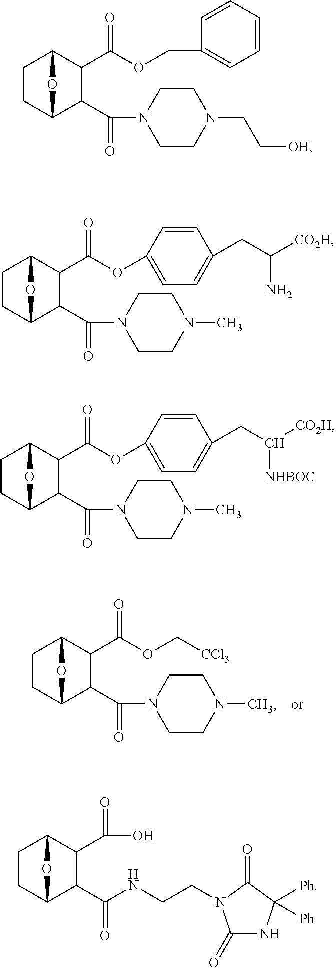

In one embodiment of the method, the protein phosphatase 2A inhibitor has the structure

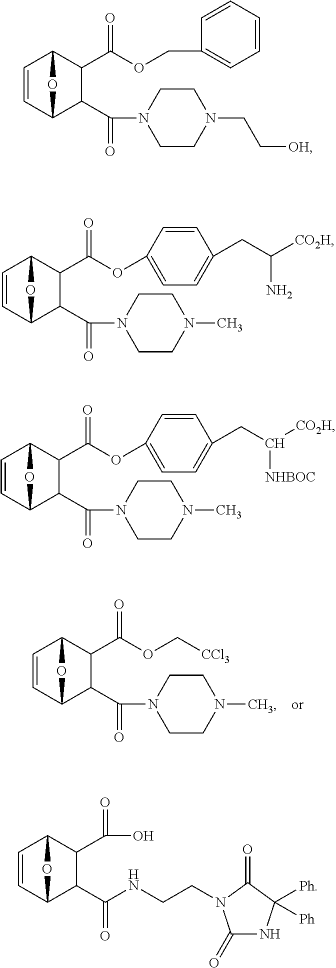

For the foregoing embodiments, each embodiment disclosed herein is contemplated as being applicable to each of the other disclosed embodiments. Thus, all combinations of the various elements described herein are within the scope of the invention.

### DEFINITIONS

As used herein, and unless otherwise stated, each of the following terms shall have the definition set forth below.

In particular, the invention is directed to the treatment or prevention of reperfusion injury.

As used herein, “reperfusion injury” is tissue damage, tissue death, cell damage, cell death, vascular leakage or endothelial dysfunction caused when blood supply returns to tissue, cells or blood vessels after a period of ischemia or lack of oxygen.

As used herein, “myocardial infarction” (MI), also known as a heart attack, is an infarction of the heart, causing cardiac tissue damage. This is most commonly due to occlusion (blockage) of a coronary artery following the rupture of a vulnerable atherosclerotic plaque, which is an unstable collection of lipids (fatty acids) and white blood cells (especially macrophages) in the wall of an artery. The resulting ischemia (restriction in blood supply) and oxygen shortage, if left untreated for a sufficient period of time, can cause damage or death of heart muscle tissue (myocardium) due to reperfusion injury.

Examples of conditions caused by ischemia and that result in reperfusion injury include, but are not limited to, myocardial infarction; cerebral infarction (stroke) due to a disturbance in the blood vessels supplying blood to the brain; pulmonary infarction or lung infarction; Splenic infarction occurs when the splenic artery or one of its branches are occluded, for example by a blood clot; Limb infarction caused by arterial embolisms; skeletal muscle infarction caused by diabetes mellitus; bone infarction; testicle infarction; and sepsis.

As used herein, a “symptom” associated with reperfusion injury includes any clinical or laboratory manifestation associated with reperfusion injury and is not limited to what the subject can feel or observe.

As used herein, “treatment of the diseases” or “treating”, e.g. of reperfusion injury, encompasses inducing prevention, inhibition, regression, or stasis of the disease or a symptom or condition associated with the disease.

As used herein, “inhibition” of disease progression or disease complication in a subject means preventing or reducing the disease progression and/or disease complication in the subject.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2, . . . n−1 or n carbons in a linear or branched arrangement, and specifically includes methyl, ethyl, propyl, butyl, pentyl, hexyl, and so on. An embodiment can be C₁-C₁₂ alkyl. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-Cₙ alkyl as defined hereinabove. The substituted amines may be substituted with alkyl, alkenyl, alkynl, or aryl groups as hereinabove defined.

The alkyl, alkenyl, alkynyl, and aryl substituents may be unsubstituted or unsubstituted, unless specifically defined otherwise. For example, a (C₁-C₆) alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, and alkynyl groups can be further substituted by replacing one or more hydrogen atoms by non-hydrogen groups described herein to the extent possible. These include, but are not limited to, halo, hydroxy, mercapto, amino, carboxy, cyano and carbamoyl.

The term “substituted” as used herein means that a given structure has a substituent which can be an alkyl, alkenyl, or aryl group as defined above. The term shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, a “compound” is a small molecule that does not include proteins, peptides or amino acids.

As used herein, an “isolated” compound is a compound isolated from a crude reaction mixture or from a natural source following an affirmative act of isolation. The act of isolation necessarily involves separating the compound from the other components of the mixture or natural source, with some impurities, unknown side products and residual amounts of the other components permitted to remain. Purification is an example of an affirmative act of isolation.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraocularly, via local delivery, subcutaneously, intraadiposally, intraarticularly, intrathecally, into a cerebral ventricle, intraventricularly, intratumorally, into cerebral parenchyma or intraparenchchymally.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

Other injectable drug delivery systems include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The compounds used in the method of the present invention may be in a salt form. As used herein, a “salt” is a salt of the instant compounds which has been modified by making acid or base salts of the compounds. In the case of compounds used to treat an infection or disease, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, an “amount” or “dose” of an agent measured in milligrams refers to the milligrams of agent present in a drug product, regardless of the form of the drug product.

As used herein, the term “therapeutically effective amount” or “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

Where a range is given in the specification it is understood that the range includes all integers and 0.1 units within that range, and any sub-range thereof. For example, a range of 77 to 90% is a disclosure of 77, 78, 79, 80, and 81% etc.

As used herein, “about” with regard to a stated number encompasses a range of +one percent to −one percent of the stated value. By way of example, about 100 mg/kg therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101 mg/kg. Accordingly, about 100 mg/kg includes, in an embodiment, 100 mg/kg. It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Synthesis of LB-107

LB-107 (5) was prepared by reacting acid 3 with N-methylpiperizine (4) in the presence of EDC. In order to prepare 5 in better yields three different methods were attempted. In the first method, one pot reaction on LB-100 using thionyl chloride in methanol was attempted but no product was observed. In the second method, acid chloride of LB-100 was allowed to react with methanol in presence of triethylamine/DMAP to give the desired methyl ester. The methyl ester thus obtained was in low yields and the separation of triethylamine from the product was also tedious. Hence a two-step procedure was used. In this third method, endothal (1) when heated under reflux in methanol gave the desired monomethylester 3 in 95% yields. Compound 3 when treated with N-methylpiperazine (4) in presence of EDC and a catalytic amount of N-hydroxybenzotriazole gave the required methyl ester 5 in 39% yields after purification with column chromatography.

### 7-Oxa-bicyclo[2,2,1]heptane-2,3-dicarboxylic acid monomethyl ester (3)

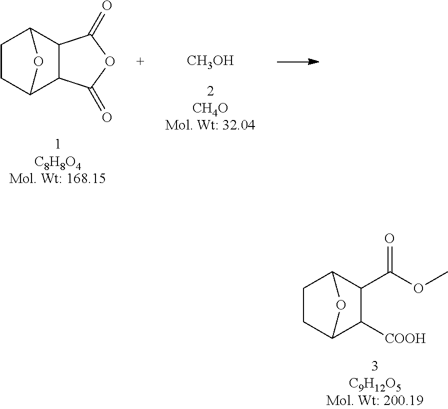

The mixture of exo-7-Oxabicyclo[2.2.1]heptane-2,3-dicarboxylic anhydride (1, 10.0 g, 59.5 mmole) and dry methanol (2, 50 mL) was heated at reflux temperature for 3.5 h. The reaction mixture became homogeneous during the reflux. The reaction mixture was then cooled down to room temperature and concentrated to give 3 (11.3 g, 95%) as crystalline white material. The crude ¹H NMR was clean enough with no extra peaks. Hence this material was utilized in the next step without further purification. ¹H NMR (DMSO-d₆) δ 1.52 (m, 4H), 2.98 (s, 2H), 3.49 (s, 3H), 4.66 (d, 2H), 12.17 (s, 1H).

### 3-(4-Methylpiperazine-1-carbonyl)-7-oxa-bicyclo[2,2,1]heptane-2-carboxylic acid methyl ester (5)

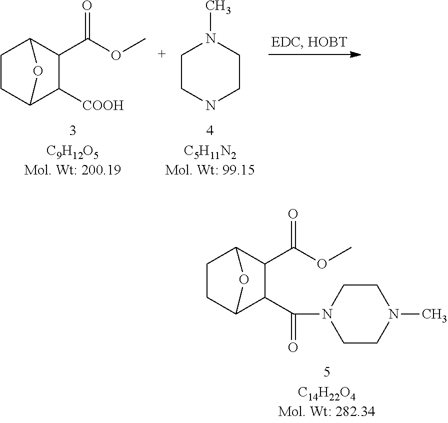

To a mixture of acid 3 (2.00 g, 10.0 mmole) in 50 mL of methylene chloride containing N-hydroxybenzotriazole (98.0 mg, 0.725 mmol) and EDC (2.09 g, 13.5 mmole) was added N-methylpiperazine (4, 1.45 g, 14.5 mmole) and the reaction mixture was stirred at room temperature overnight. The reaction mixture was evaporated to dryness and the product purified by column using 5% methanol in methylene chloride to give the required ester 5 as a semi solid (1.89 g, 67%). This was further purified by triturating with isopropyl ether followed by re-crystallization with a mixture of ethyl acetate/Hexane to give a white crystalline material of 5 (LB-107)(1.10 g, yield: 39%, mp 108-109° C.). The mother liquor was concentrated and saved for future recrystallization. ¹H NMR (CDCl₃) δ 1.50 (m, 2H), 1.83 (m, 2H), 2.30-2.44 (m, 7H), 2.94 (d, J=9.6 Hz, 1H), 3.10 (d, J=9.6 Hz, 1H), 3.50 (m, 3H), 3.71 (m, 4H), 4.90 (m, 2H), ESMS: 282.

### Example 2

### Protein Phosphatase 2A Inhibitors

The compounds used in the method of the present invention are protein phosphatase 2A (PP2A) inhibitors (Lu et al., 2009; U.S. Pat. No. 7,998,957 B2). Compounds LB-100 and LB-102 are inhibitors of PP2A in vitro in human cancer cells and in xenografts of human tumor cells in mice when given parenterally in mice. These compounds inhibit the growth of cancer cells in mouse model systems. It has also been shown that another structural homolog of these compounds, LB-107, is active when given orally to mice.

LB100, LB102 or LB107 are tested in an animal model of cardiac ischemia-reperfusion injury.

The structure of LB100 is:

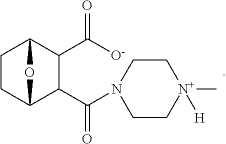 [SMILES: C[NH+]1CCN(C(=O)C2C(C(=O)[O-])[C@H]3CC[C@@H]2O3)CC1]

The structure of LB102 is:

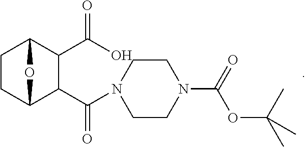 [SMILES: CC(C)(C)OC(=O)N1CCN(C(=O)C2C(C(=O)O)[C@H]3CC[C@@H]2O3)CC1]

The structure of LB107 is:

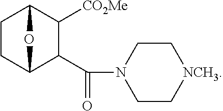 [SMILES: COC(=O)C1C(C(=O)N2CCN(C)CC2)[C@@H]2CC[C@H]1O2]

### Example 3

### Increase Phosphorylation and Activation of Akt

Compounds LB-100, LB-102, LB-107, and other homlogs of LB-100 disclosed herein increase phosphorylation of Akt in mammalian cells, including, but not limited to, cardiac cells, brain cells and endothelial cells. Compounds LB-100, LB-102 and LB-107 and other homologs of LB-100 disclosed herein reduce dephosphorylation and inactivation of Akt by protein phosphatase 2A (PP2A) in mammalian cells, including, but not limited to, cardiac cells, brain cells and endothelial cells. Compounds LB-100, LB-102 and LB-107 and other homologs of LB-100 disclosed herein increase activation of Akt by protein phosphatase 2A (PP2A) in mammalian cells, including, but not limited to, cardiac cells, brain cells and endothelial cells

### Example 4

### Reperfusion Injury

Compounds LB-100, LB-102, LB-107 and other homologs of LB-100 disclosed herein reduce reperfusion injury in mammalian tissue that has suffered from an ischemia. The mammalian tissue includes, but is not limited to, cardiac tissue, brain tissue and endothelial tissue.

### Example 5

### Myocardial Infarction

Compounds LB-100, LB-102, LB-107 and other homlogs of LB-100 disclosed herein reduce tissue damage associated with reperfusion injury in the heart of a subject following a myocardial infarction. Compounds LB-100, LB-102, LB-107 and other homlogs of LB-100 disclosed herein prevent tissue damage associated with reperfusion injury in the heart of a subject following a myocardial infarction.

### Example 5

### Sepsis

Compounds LB-100, LB-102, LB-107 and other homlogs of LB-100 disclosed herein reduce vascular leakage associated with reperfusion injury in a subject suffering from sepsis. Compounds LB-100, LB-102, LB-107 and other homlogs of LB-100 disclosed herein reduce endothelial dysfunction associated with reperfusion injury in a subject suffering from sepsis.

### Example 6

### Study of LB-100 to Improve Vascular Integrity and Reduce Tissue Damage Following Acute Trauma to Tissue

The ability of LB-100 to improve vascular integrity and to reduce tissue damage following acute trauma to tissue was tested by analyzing the survival of tissue at the distal end of a flap graft in in-bred rats infused with LB-100 via an implanted pump prior to raising a skin flap.

An ALZET pump (model 1007D, reservoir volume 100 ul) was implanted subcutaneously under anesthesia subcutaneously four days before creating a skin flap again under anesthesia. The pump in control animals contained sodium chloride and the pump in treated animals contained 0.55 mg of LB-100. The pump delivers 0.5 ul/hour+/−10% so each animal received about 12 ul/day for 8 days, 4 days prior to raising the graft and for the first 4 days after creation of the graft. There were 10 animals in each group.

The animals were sacrificed on day 7 following creation of the flap, the amount of necrosis at the end of the flap was measured by planimetry. The area of necrosis at the end of the flap was divided by total surface area of the entire flap and expressed as a percentage. Table 1 shows the area (%) of necrosis in control rats 1-10 vs rats receiving LB-100 (treatment rats).

TABLE 1
Area of necrosis in control rats vs rats receiving LB-100.

| Control | Total | Necrosis | % | Treatment | Total | Necrosis | % |
|---|---|---|---|---|---|---|---|
| 1 | 245751 | 139686 | 56.8404605 | 1 | 180926 | 55473 | 30.6606016 |
| 2 | 222271 | 94119 | 42.3442554 | 2 | 188880 | 63777 | 33.7658831 |
| 3 | 273475 | 142373 | 52.0607002 | 3 | 254862 | 66144 | 25.9528686 |
| 4 | 293026 | 87832 | 29.974132 | 4 | 154749 | 37181 | 24.0266496 |
| 5 | 265486 | 124567 | 46.9203649 | 5 | 227395 | 77089 | 33.9009213 |
| 6 | 227431 | 73396 | 32.2717659 | 6 | 200725 | 64147 | 31.9576535 |
| 7 | 269259 | 118707 | 44.0865486 | 7 | 231616 | 66886 | 28.8779704 |
| 8 | 301765 | 124054 | 41.1094726 | 8 | 251903 | 77327 | 30.6971334 |
| 9 | 223558 | 78105 | 34.9372422 | 9 | 231841 | 91515 | 39.4731734 |
| 10 | 248844 | 74858 | 30.0823006 | 10 | 277689 | 112275 | 40.431922 |
| | | Average | 41.0627243 | | | Average | 31.9744777 |

Analysis of the planimetry results revealed that the mean decrease in extent of necrosis in treated animals was 22%.

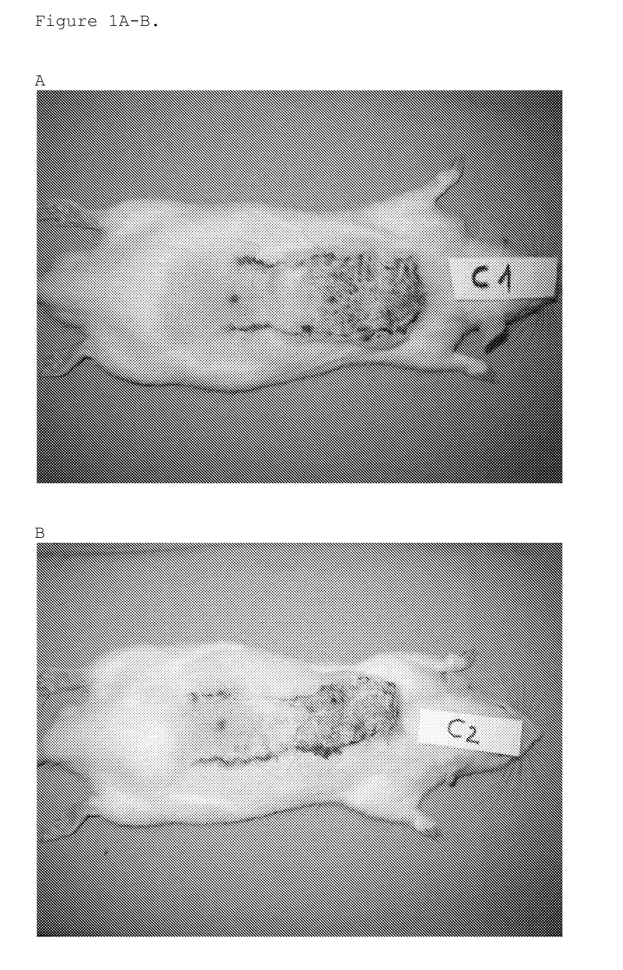

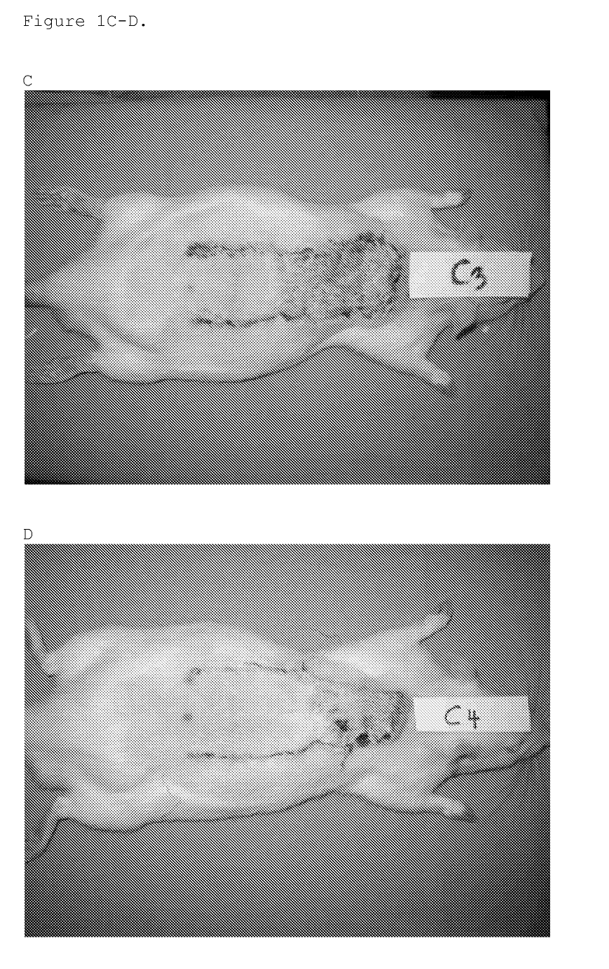

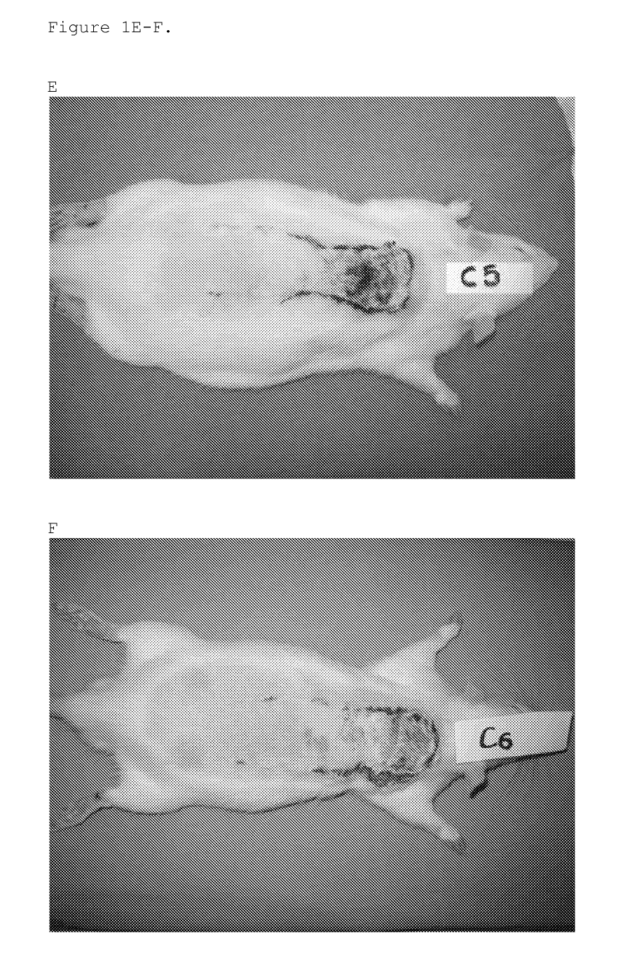

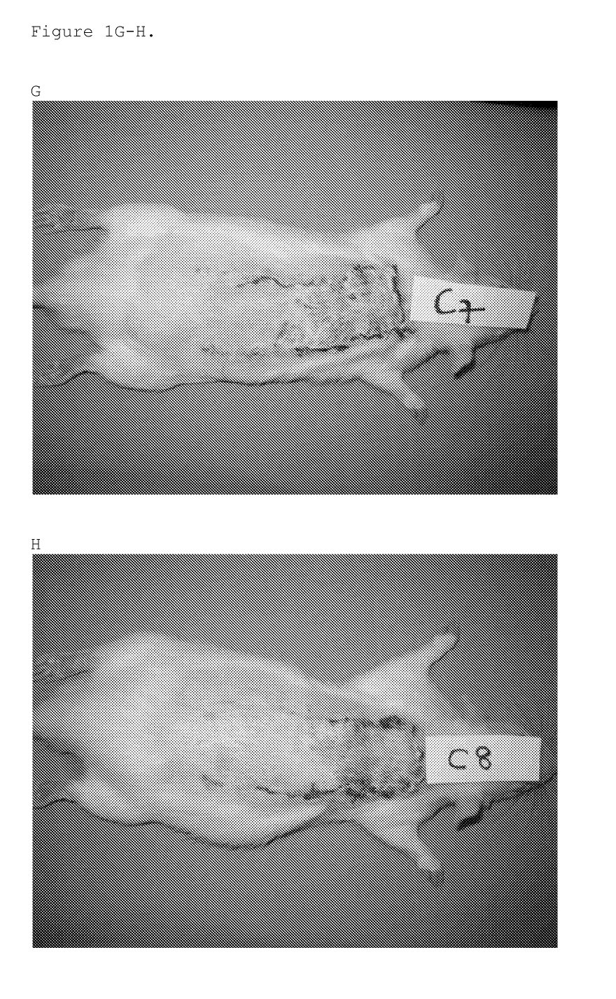

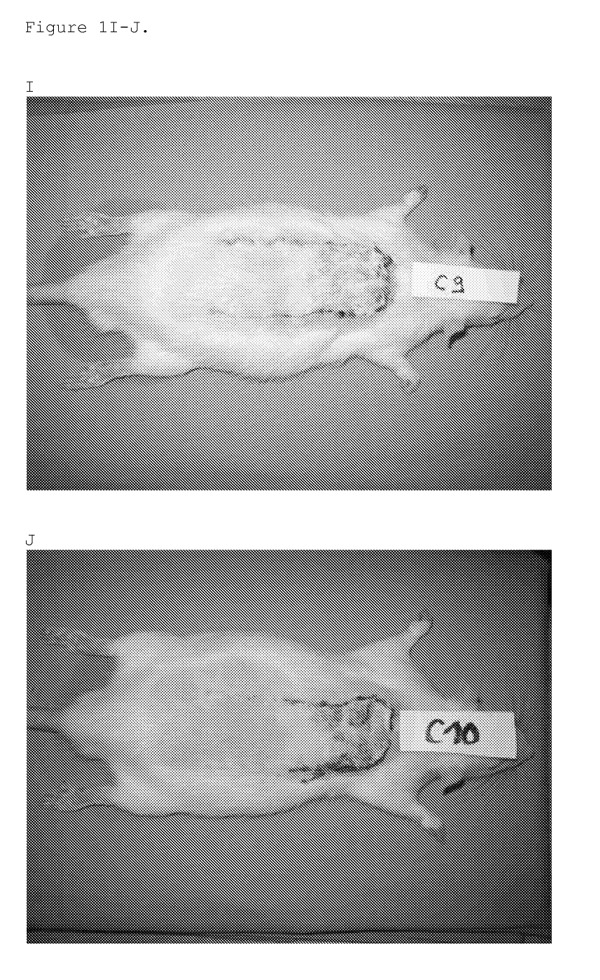

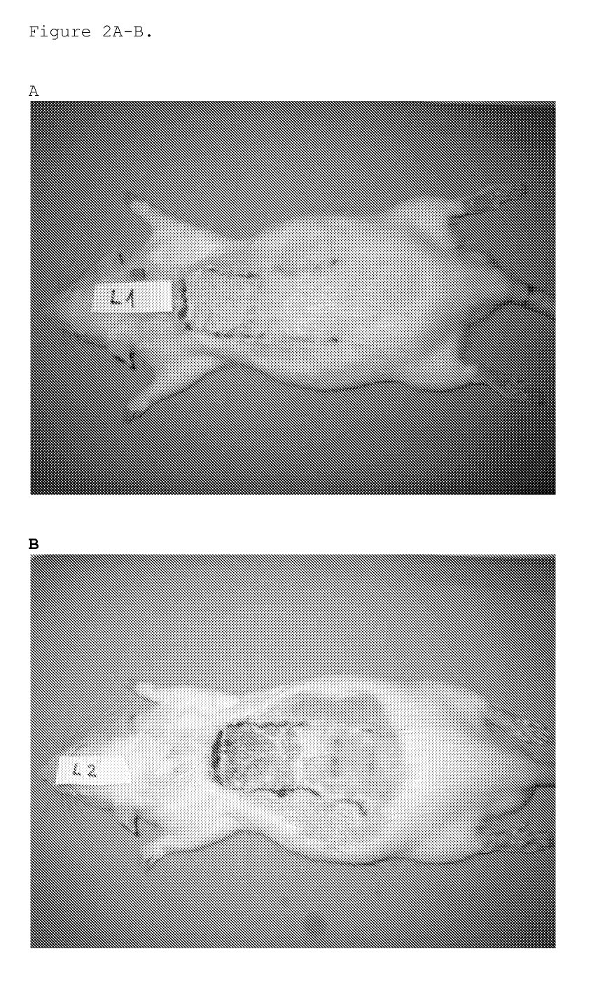

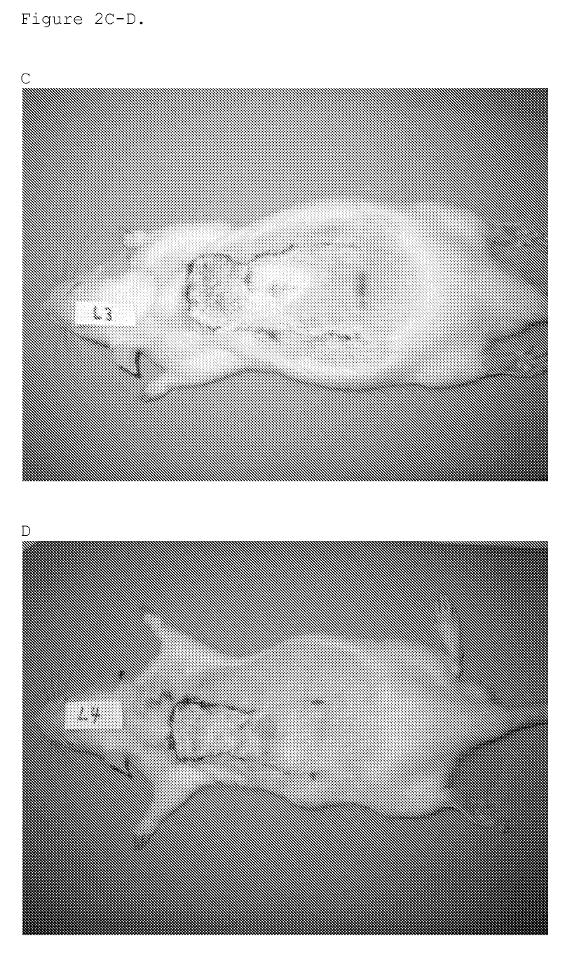

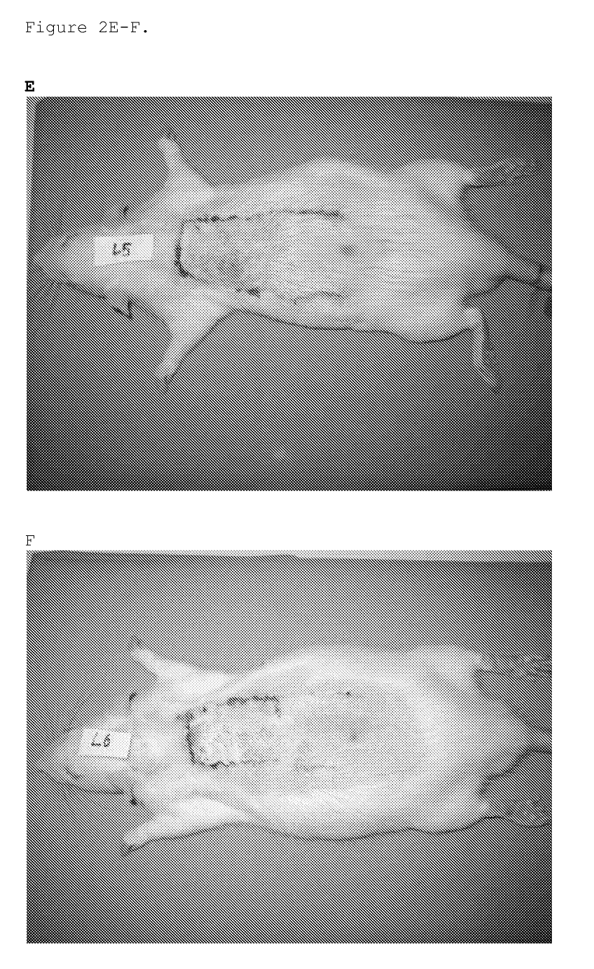

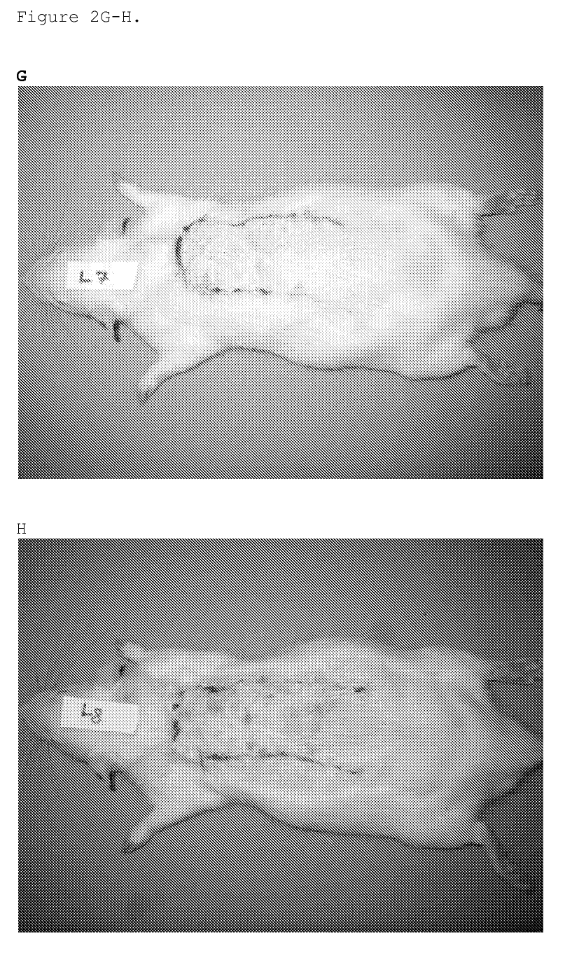

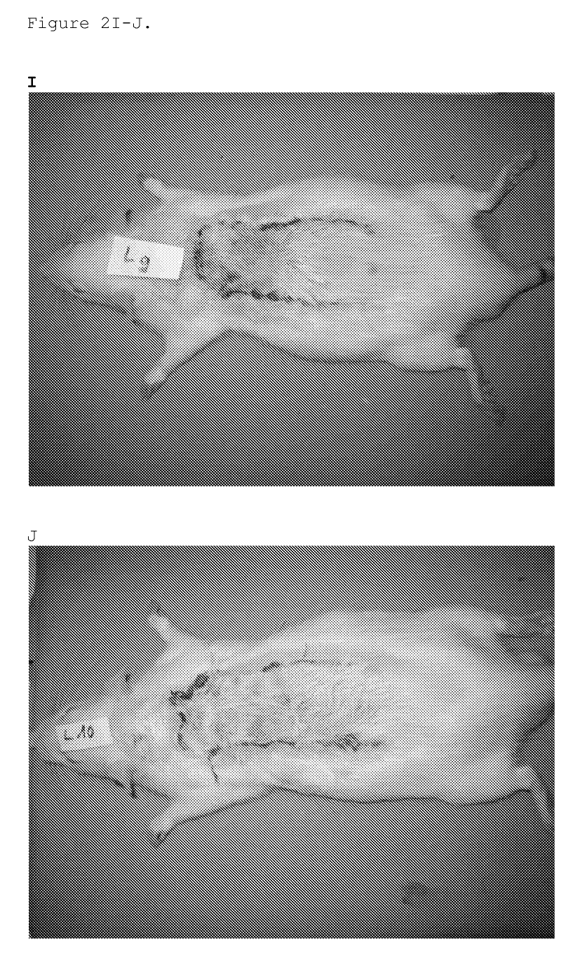

The visual appearance of the flaps was also inspected. FIGS. 1a-j show control rats 1-10 on day 7 and FIGS. 2a-j show treatment rats 1-10 on day 7. The treatment rats had received LB-100.

As shown in the photographs of the flaps on day 7 (FIGS. 1a-j and 2a-j), there was less extensive necrosis at the distal ends of the flap in those animals receiving LB-100.

### Discussion

It has been known since the early 1970s (Maroko et al, 1971) that brief episodes of oxygen deprivation (ischemia) and relief of the ischemia (reperfusion) protects the heart against damage from a subsequent episode. In 1986, Murray et al showed that several short periods of cardiac ischemia reduced the size of tissue damage (infarct) following prolonged ischemia in a canine model. In 2003, Zhao et al. demonstrated that several rounds of ischemia and the return of blood flow (reperfusion) following the induction of an experimental heart attack, also reduced the size of the infarct, compared to simple reperfusion after the attack. The mechanism(s) responsible for cardiac protection by pre- or post-brief cycles of ischemia has been the subject of more than 2500 papers and, although the phenomenon is demonstrable in several animal models, remains unclear. Despite a lack of understanding of the molecular basis of pharmacologic cardioprotection, however, recently there have been increasing calls for finding a way to reduce the amount of cardiac tissue damage following a heart attack (Cohen and Downey, 2011).

In 1998, Weinbrenner et al. reported that fostriecin (FOS), an inhibitor of protein phosphatase 2A, minimizes the size of infarction of cardiac tissue (myocardium) before and even when given after the onset of oxygen deprivation. The following year, Barancik et al. (1999) showed that another known inhibitor of serine/threonine phosphatase, okadaic acid, protected pig myocardium against ischemia (reduced the size of infracted tissue) in an in vivo model and also showed that the activity of phosphatases, especially that of PP2A, were decreased in the heart tissue infused with the inhibitor.

FOS, a well studied inhibitor of PP2A, reduces injury even when given after the onset of coronary artery restriction and confirmation of this phenomenon by Barancik et al. (1999) using another PP2A inhibitor, okadaic acid in a pig heart model. Weinbrenner et al. (1998) studied a rabbit and pointed out that death of tissue in the rabbit heart progresses about 5 times faster than in the primate heart, leading them to speculate that administration of a PP2A inhibitor such as FOS as late as 50 minutes after the start of heart attack signs in humans might be expected to offer protection. In earlier studies, Armstrong et al (Armstrong et al. 1997; Armstrong et al. 1998) demonstrated that protein phosphatase inhibitor calyculin A, a PP1/2A inhibitor and FOS both afforded protection from injury to rabbit and pig cells in vitro. Subsequently, Fenton et al. (2005) in rats showed that okadaic acid reduced cellular death following induced MI in both young and aged animals. Fan et al. (2010) studied the effects of okadaic acid at concentrations inhibiting only PP2A and the effects of cantharadin, a naturally occurring inhibitor of both PP1 and PP2A, in isolated perfused functioning rat hearts and concluded that the use of phosphatase inhibitors may provide an approach to reducing reperfusion-induced cell death.

The mechanism by which PP2A confers protection of myocardial damage due to oxygen deprivation is believed to be via activation of a major cell signaling pathway, the PI3K-Akt pathway (Matsui et al. 2001). Recently, Kunuthur (Kunuthur et al. 2011) showed that of three Akt isoforms, Akt1 is essential for cardioprotection against ischemic-reperfusion injury.

Inhibition of PP2A by the novel inhibitors LB-100 and LB-102 and other structural homologs of these compounds have been shown to result in increased phosphorylation of Akt (Lu et al. 2009; U.S. Pat. No. 8,0858,268). Phosphorylation of Akt leads to its activation, which in turn increases the phosphorylation of several proteins affecting mitochondrial function and mediating cell death (Tsang et al. 2005).

Without wishing to be bound by any scientific theory, Akt mediates protection by phosphorylation of a number of target proteins, including GSK-31, endothelial nitric oxide synthase (eNOS), the proapoptotic Bcl-2 family member BAD, caspase 9, the ubiquitin ligase murine double minute 2 (mdm2), and others (Fayard, E. et al, 2005). Phosphorylation of BAD suppresses apoptosis and promotes cell survival (Datta, S. R. et al. 1997). Overexpression of Akt blocks hypoxia-mediated activation of caspase 9, also blocking their proapoptotic roles (Uchiyama, T. et al. 2004). Akt phosphorylates and activates the ubiquitin ligase, mdm2, which has been shown to play a role in reducing hypoxia-reoxygenation cell death in myocytes (Toth, A. 2006). Akt phosphorylates and activates eNOS, resulting in an increase in nitric oxide (NO) production, which may activate a number of pathways resulting in cardiprotection (Tong, H. et al.). Akt also phosphorylates and inactivates GSK-3β, which also provides an anti-apoptotic effect (Tong et al. 2000).

LB-100, LB-102, and LB-107 and other structural homologs are effective inhibitors of PP2A. LB-102, for example, inhibits PP2A with IC50 of about 0.4 uM and to much lesser extent PP1 with an IC50 of about 8.0 uM (Lu et al. 2009(a)). LB-100 is currently entering a Phase I clinical trial in which the compound is anticipated to enhance the cytotoxicity of DNA damaging agents. Although the maximum tolerated dose in humans has not yet been determined, toxicokinetic studies in rats and dogs indicate that the compound can be given with acceptable and reversible toxicity at doses known to inhibit PP2A in tissue of rodents. Thus, LB-100, LB-102 and/or structural homologs can be safely administered to human beings to minimize the extent of myocardial damage following MI. Administration of PP2A is also expected to reduce the extent of tissue damage caused by ischemia in other disease states, such as stroke and acute tissue injury due to trauma, either accidental or surgical injury that compromises blood supply to tissue acutely.

Tissue ischemia also results from hypotension secondary to acute bacterial infections. PP2A is activated due to the inflammatory processes, particularly sepsis, and treatment with LB-100 and structural homologs may be highly beneficial, indeed lifesaving. Sepsis leads to activation of NADPH oxidase and uncoupling of endothelial nitric oxide synthase to produce superoxide, increased NO production and neuronal NOS activity, with increased 3-nitrotyrosine formation and increase PP2A activity in the hind limbs of animals (Zhou et al. 2012). Zhou demonstrated that rapid injection of ascorbate protected against these effects including reduction of PP2A activation. This same mechanism was found by Ladurner (Ladurner et al. 2012). Ladurner showed that in addition to ascorbate, the protein phosphatase inhibitor okadaic acid had similar effects supporting the hypothesis that endothelial damage in these model systems is mediated by PP2A. Han (Han et al. 2010) had previously demonstrated that okadaic acid at a concentration that inhibits PP2A activity decreases endothelial barrier disruption caused by septic insult. Wu and Wilson (Wu et al. 2009) studied mouse skeletal muscle endothelial cells and showed that PP2A inhibition by okadaic acid preserved endothelial barrier function. Thus, LB-100 is expected to be useful in the treatment of conditions such as septic shock in which PP2A mediates vascular leakage.

One mechanism by which fostriecin and the LB-100 compounds reduce tissue damage following acute trauma to tissue may be by inhibition of PP2A in the vasculature. The ability of LB-100 to improve vascular integrity and to reduce tissue damage following acute trauma to tissue was tested by analyzing survival of tissue at the distal end of a flap graft as described in Example 6. The data from Example 6 show that the mean decrease in extent of necrosis in treated animals was 22%. Such an improvement in graft survival would be valuable clinically where necrosis of the distal end of a flap graft is a major limitation to this surgical intervention to correct tissue defects.

### REFERENCES

Armstrong S C et al (1997) Comparison of in vitro pre-conditioning responses of isolated pig and rabbit cardiomyocytes: effects of a protein phosphatase inhibitor fostriecin. J Mol Cell Cardiol 29:3009-3024.
Armstrong S C et al (1998) Protein phosphates inhibitors calyculin A and fostriecin protect rabbit cardiomyocytes in late ischemia. J Mol Cell Cardiol 30:61-73.
Barancik M et al (1999) Okadaic acid and anisomycin are protective and stimulate the SAPK/JNK pathway. Journal of Cardiovascular Pharmacology 34(2):182-190.
Cohen M V and Downey J M (2011) Is it time to translate ischemic preconditioning's mechanism of cardioprotection into clinical practice?J of Cardiovascular Pharmacology and Therapeutics 16(3-4):273-280.
Datta, S. R. (1997) Akt Phosphorylation of BAD Couples Survival Signals to the Cell-Intrinsic Death Machinery. Cell, 91, 231-241.
Fan W J (2010) Kinases and phosphatases in ischaemic preconditioning: a re-evaluation. Basic RES Cardiol 105:495-511.
Fenton et al (2005). Inhibition of phosphatase activity enhances pre-conditioning and limits cell death in the ischemic/reperfused aged rat heart. Life Sciences 77:3375-3388.
Fayard E, Tintignac L A, Baudry A, Hemmings B A. (2005) Protein kinase B/Akt at a glance. J Cell Sci 118: 5675-5678.
Kunuthur S P et al. (2011) The Akt1 isoform is an essential mediator of ischemic preconditioning. Journal of Cellular and Molecular Medicine. “Accepted Article”; doi:10.1111/j.1582-4934.2011.01491.x
Ladurner et al. (2012) Ascorbate stimulates endothelial nitric oxide synthese enzyme activity by rapid modulation of its phosphorylation status. Free Radical Biology and Medicine, In Press.
Longacre L S et al (2011) New horizons in cardio protection. Circulation 124:1172-1179.
Lu J et al (2009) The effect of a PP2A inhibitor on the nuclear receptor corepressor pathway in glioma. Journal of Neurosurgery DOI:10.3171/2009.11 JNS091272.
Lu J et al (2009) (a) Inhibition of serine/threonine phosphatase PP2A enhances cancer chemotherapy by blocking DNA damage induced defense mechanisms. PNAS 106(28):11697-11702.
Maroko P R et al (1971) Factors influencing infarct size following experimental coronary artery occlusions. Circulation 43:67-82.
Matsui T et al (2001). Akt activation preserves cardiac function and prevents injury after transient carciac ischemia in vivo. Circulation 104:330-335.
U.S. Pat. No. 8,058,268 B2. Neuroprotective agents for the prevention and treatment of neurodegenerative diseases.
Tsang A et al. (2005) Myocardial postconditioning: reperfusion injury revisited. American Journal of Physiology Heart Circulation Physiology 289: H2-H7.
Tong H, Chen W, Steenbergen C, Murphy E. (2000) Ischemic preconditioning activates phosphatidylinositol-3-kinase upstream of protein kinase C. Circ Res 87: 309-315.
Toth A, Nickson P, Qin L L, Erhardt P. (2006) Differential regulation of cardiomyocyte survival and hypertrophy by MDM2, an E3 ubiquitin ligase. J Biol Chem 281: 3679-3689.
Uchiyama T, Engelman R M, Maulik N, Das D K. (2004) Role of Akt signaling in mitochondrial survival pathway triggered by hypoxic preconditioning. Circulation 109: 3042-3049.
Weinbrenner C et al (1998) Fostriecin, an inhibitor of phosphatase 2A, limits myocardial infarct size even when administered after onset of ischemia. Circulation 98:899-905.
Wu, et al. (2007) Activation or Protein Phosphatase 2A by Palmitate Inhibits AMP-activated Protein Kinase. The Journal of Biological Chemistry. 282, 13, 9777-9788.
Yellon D M and Hausenloy D J (2005) Realizing the clinical potential of ischemic preconditioning and postconditioning. Nature Clinical Practice Cardiovascular Medicine 2(11)568-575.
Zhou, G. et al. (2012) Ascorbate protects against vascular leakage in cecal ligation and puncture-induced septic peritonitis. American J Physiol Regul Integr Comp Physiol 302:409-416.
Zhao Z-Q et al (2003) Inhibition of myocardial injury by ischemic postconditioning during reperfusion: comparison with preconditioning. American Journal of Physiology 285(2):H579-H588.

1. A method of reducing reperfusion injury in mammalian tissue comprising contacting the tissue with a protein phosphatase 2A (PP2A) inhibitor having the structure: 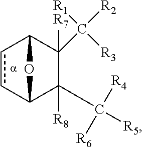
wherein
bond α is present or absent;
R₁ and R₂ is each independently H, O⁻ or OR₉,
where R₉ is H, alkyl, alkenyl, alkynyl or aryl,
or R₁ and R₂ together are ═O;
R₃ and R₄ are each different, and each is OH, O⁻, OR₉, O(CH₂)₁₋₆R₉, SH, S⁻, SR₉, 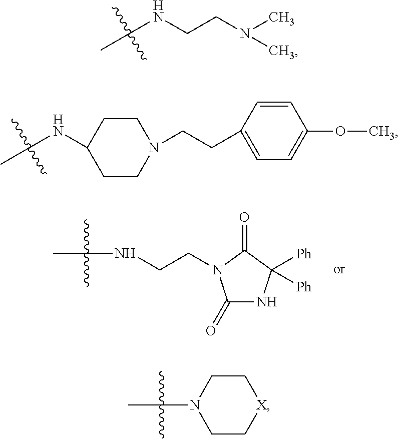
where X is O, S, NR₁₀, or N⁺R₁₀R₁₀,
where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O, 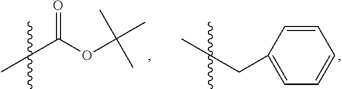
—CH₂CN, —CH₂CO₂R₁₁, —CH₂COR₁₁, —NHR₁ or —NH⁺(R₁₁)₂,
wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
where R₁₂ is H, alkyl, alkenyl, alkynyl or aryl; and
each occurrence of alkyl, alkenyl, or alkynyl is branched or unbranched, unsubstituted or substituted,
or a salt, enantiomer or zwitterion of the compound.

2. The method of claim 1, wherein the reduction of reperfusion injury comprises increased phosphorylation of Akt in the mammalian tissue that has suffered an ischemia.

3. The method of claim 2, wherein the reduction of reperfusion injury comprises increased activation of Akt in the mammalian tissue that has suffered an ischemia.

4. The method of claim 1, wherein the reduction of reperfusion injury comprises increased phosphorylation of BAD, mdm2, eNOS and/or GSK-3β in the mammalian tissue that has suffered an ischemia.

5. The method of claim 1, wherein the ischemia is caused by a myocardial infarction, stroke or sepsis.

6. The method of claim 1, wherein the tissue is myocardial tissue, brain tissue or endothelial tissue.

7. The method of claim 1, wherein the protein phosphatase 2A inhibitor has the structure 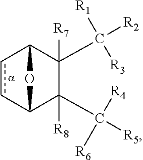
wherein
bond α is present or absent;
R₁ and R₂ is each independently H, O⁻ or OR₉,
where R₉ is H, alkyl, alkenyl, alkynyl or aryl,
or R₁ and R₂ together are ═O;
R₃ and R₄ are each different, and each is OH, O⁻, OR₉, SH, S⁻, SR₉, 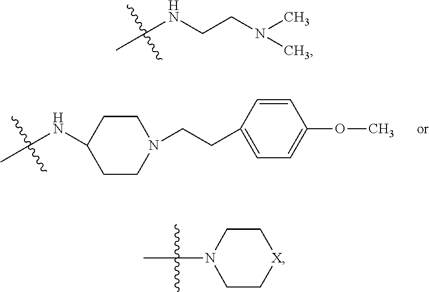
where X is O, S, NR₁₀, or N⁺R₁₀R₁₀,
where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O, 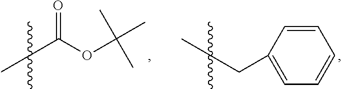
—CH₂CN, —CH₂CO₂R₁₁, —CH₂COR₁₁, —NHR₁₁ or —NH⁺(R₁₁)₂,
where each R₁₁ is independently alkyl, alkenyl alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
where R₁₂ is H, aryl or a substituted or unsubstituted alkyl, alkenyl or alkynyl; and
each occurrence of alkyl, alkenyl, or alkynyl is branched or unbranched, unsubstituted or substituted,
or a salt, enantiomer or zwitterion of the compound.

8-12. (canceled)

13. The method of claim 7,
wherein
R₁ and R₂ together are ═O;
R₃ is O⁻ or OR₉,
where R₉ is H, methyl, ethyl or phenyl;
R₄ is 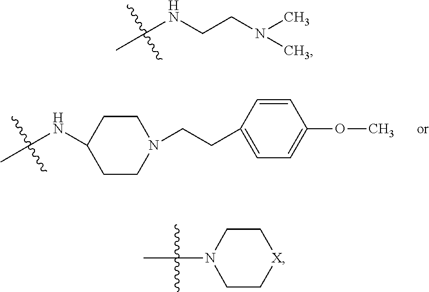
where X is O, S, NR₁₀, or N⁺R₁₀R₁₀,
where each R₁₀ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro, 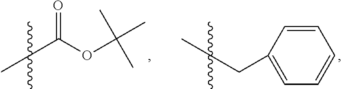
—CH₂CN, —CH₂CO₂R₁₁, —CH₂COR₁₁, —NHR₁₁ or —NH⁺(R₁₁)₂,
where R₁₁ is alkyl, alkenyl or alkynyl, each of which is substituted or unsubstituted, or H;
R₅ and R₆ taken together are ═O; and
R₇ and R₈ is each independently H, F, Cl, Br, SO₂Ph, CO₂CH₃, or SR₁₂,
where R₁₂ is a substituted or unsubstituted alkyl, alkenyl or alkynyl.

14-17. (canceled)

18. The method of claim 13,
wherein R₄ is 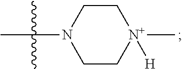 [SMILES: *N1CC[NH+](C)CC1]
or
wherein R₄ is 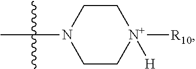
where R₁₀ is 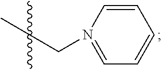
or
wherein R₄ is 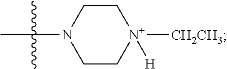 [SMILES: *N1CC[NH+](CC)CC1]
or
wherein R₄ is H.

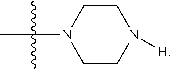

19-35. (canceled)

36. The method of claim 7, wherein the protein phosphatase 2A inhibitor has the structure 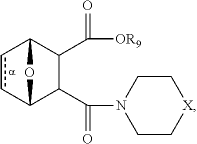
wherein
bond α is present or absent;
R₉ is present or absent and when present is H, alkyl, alkenyl, alkynyl or phenyl; and
X is O, NR₁₀, or N⁺R₁₀R₁₀,
where each R₁₀ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro, 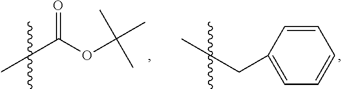
—CH₂CN, —CH₂CO₂R₁₂, or —CH₂COR₁₂,
where R₁₂ is H or alkyl,
or a salt, zwitterion, or enantiomer of the compound.

37. (canceled)

38. (canceled)

39. (canceled)

40. The method of claim 7, wherein the protein phosphatase 2A inhibitor has the structure

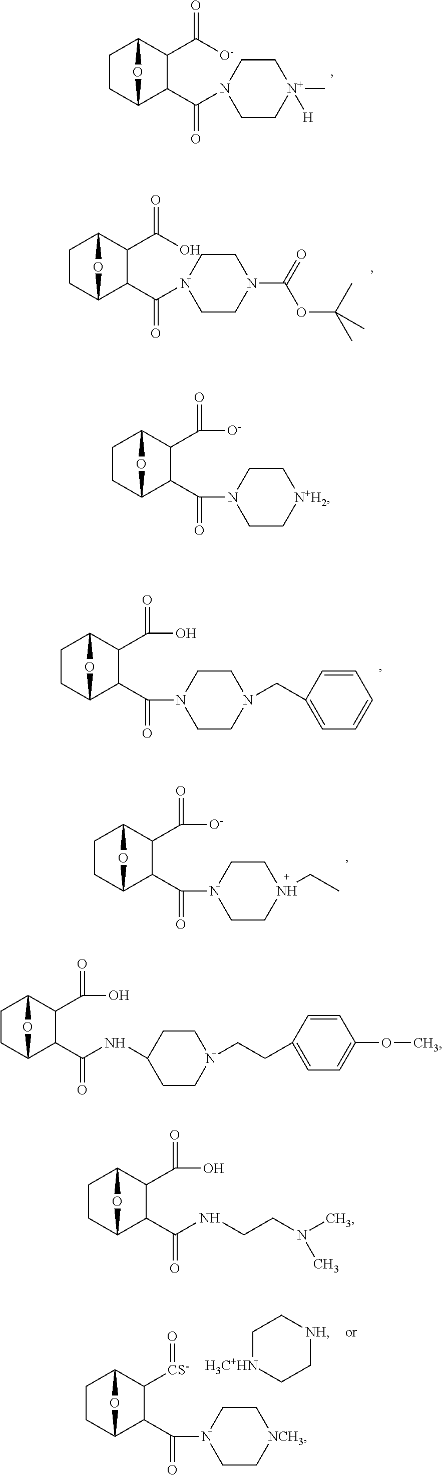

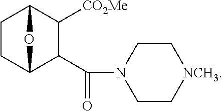 [SMILES: COC(=O)C1C(C(=O)N2CCN(C)CC2)[C@@H]2CC[C@H]1O2]

41. The method of claim 7, wherein the protein phosphatase 2A inhibitor has the structure

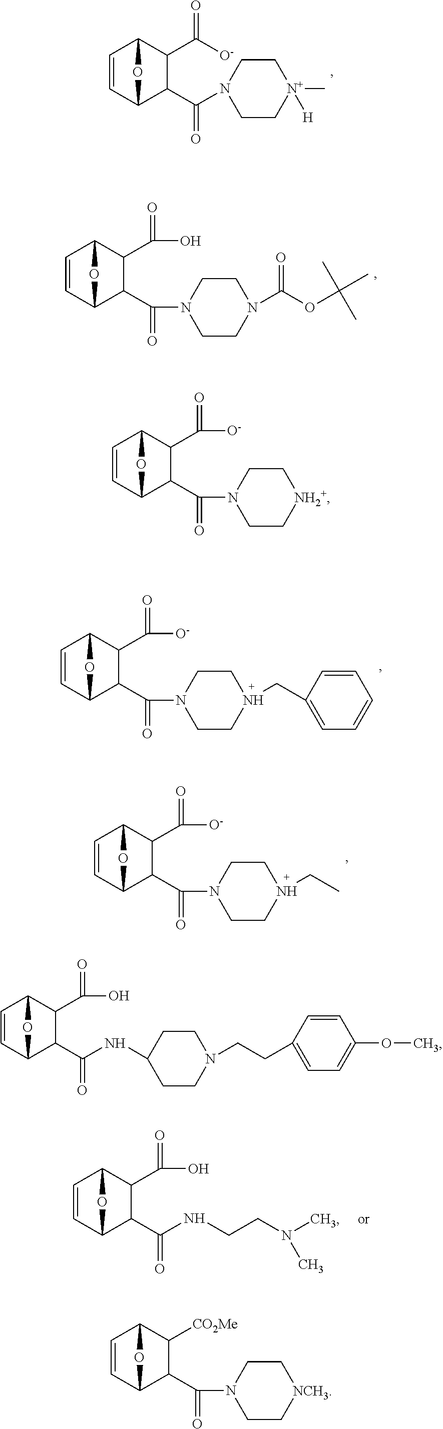

42. (canceled)

43. (canceled)

44. The method of claim 7, wherein the protein phosphatase 2A inhibitor has the structure 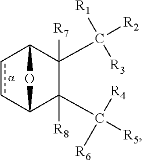
wherein
bond α is present or absent;
R₁ and R₂ is each independently H, O⁻ or OR₉,
where R₉ is H, alkyl, alkenyl, alkynyl or aryl,
or R₁ and R₂ together are ═O;
R₃ and R₄ are each different, and each is O(CH₂)₁₋₆R₉ or OR₉ or 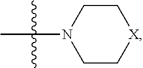
where X is O, S, NR₁₀, or N⁺R₁₀R₁₀,
where each R₁₀ is independently H, alkyl,
hydroxyalkyl, C₂-C₁₂ alkyl, alkenyl, C₄-C₁₂ alkenyl, alkynyl, aryl, where the
substituent is other than chloro when R₁ and R₂ are ═O, 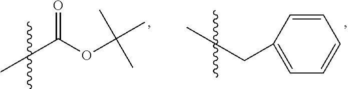
—CH₂CN, —CH₂CO₂R₁₁, —CH₂COR₁₁, —NHR₁₁ or ═NH⁺(R₁₁)₂,
where each R₁₁ is independently H, alkyl, alkenyl, or alkynyl;
or R₃ and R₄ are each different and each is OH or 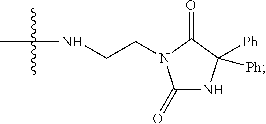 [SMILES: *NCCN1C(=O)NC(c2ccccc2)(c2ccccc2)C1=O]
R₅ and R₆ is each independently H, OH, or R₅ and R₆ taken together are ═O;
R₇ and R₈ is each independently H, F, Cl, Br, SO₂Ph, CO₂CH₃, or SR₁₂,
where R₁₂ is H, aryl or a substituted or unsubstituted alkyl, alkenyl or alkynyl; and
each occurrence of alkyl, alkenyl, or alkynyl is branched or unbranched, unsubstituted or substituted,
or a salt, enantiomer or zwitterion of the compound.

45-50. (canceled)

51. The method of claim 44,
wherein
R₁ and R₂ together are ═O;
R₃ is OH, O(CH₂)R₉, or OR₉,
where R₉ is phenyl or CH₂CCl₃, 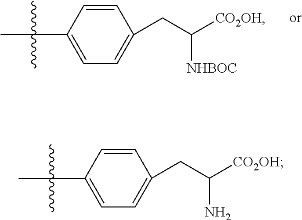
R₄ is 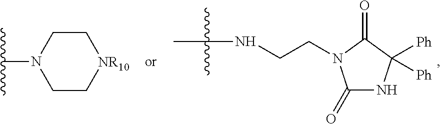
where R₁₀ is CH₃ or CH₃CH₂OH;
R₅ and R₆ together are ═O; and
R₇ and R₈ are each independently H.

52-58. (canceled)

59. The method of claim 44, wherein the protein phosphatase 2A inhibitor has the structure

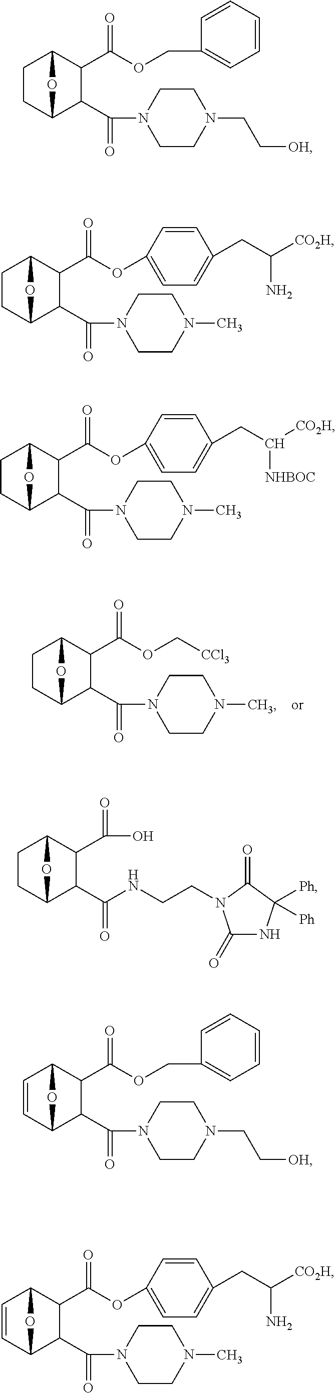

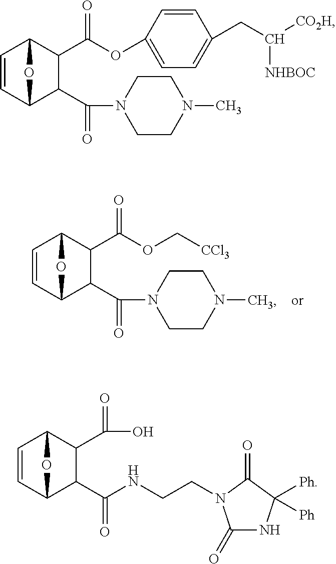

60. (canceled)

61. A method of reducing tissue damage associated with reperfusion injury in the heart of a subject following a myocardial infarction comprising administering to the subject a therapeutically effective amount of a protein phosphatase 2A (PP2A) inhibitor having the structure: 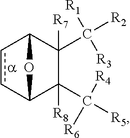
wherein
bond α is present or absent;
R₁ and R₂ is each independently H, O⁻ or OR₉,
where R₉ is H, alkyl, alkenyl, alkynyl or aryl,
or R₁ and R₂ together are ═O;
R₃ and R₄ are each different, and each is OH, O⁻, OR₉, OR₁₀, O(CH₂)₁₋₆R₉, SH, S⁻, SR₉, 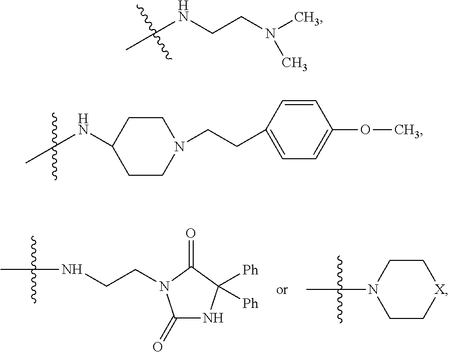
where X is O, S, NR₁₀, or N⁺R₁₀R₁₀,
where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O, 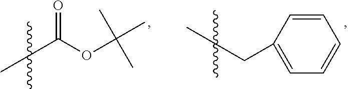
—CH₂CN, —CH₂CO₂R₁₁, —CH₂COR₁₁, —NHR₁₁ or —NH⁺(R₁₁)₂,
wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
where R₁₂ is H, alkyl, alkenyl, alkynyl or aryl; and
each occurrence of alkyl, alkenyl, or alkynyl is branched or unbranched, unsubstituted or substituted,
or a salt, enantiomer or zwitterion of the compound.

62. A method of reducing vascular leakage associated with reperfusion injury in a subject suffering from sepsis comprising administering to the subject a therapeutically effective amount of a protein phosphatase 2A (PP2A) inhibitor having the structure: 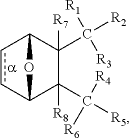
wherein
bond α is present or absent;
R₁ and R₂ is each independently H, O⁻ or OR₉,
where R₉ is H, alkyl, alkenyl, alkynyl or aryl,
or R₁ and R₂ together are ═O;
R₃ and R₄ are each different, and each is OH, O⁻, OR₉, OR₁₀, O(CH₂)₁₋₆R₉, SH, S⁻, SR₉, 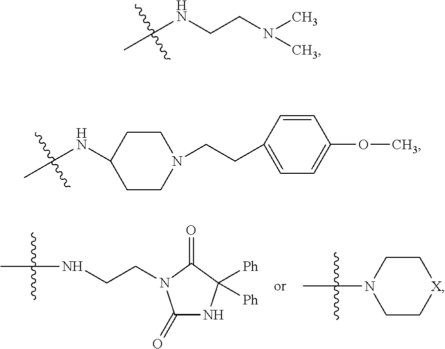
where X is O, S, NR₁₀, or N³⁰R₁₀R₁₀,
where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O, 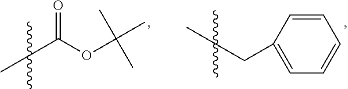
—CH₂CN, —CH₂CO₂R₁₁, —CH₂COR₁₁, —NHR₁₁ or —NH⁺(R₁₁)₂,
wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
where R₁₂ is H, alkyl, alkenyl, alkynyl or aryl; and
each occurrence of alkyl, alkenyl, or alkynyl is branched or unbranched, unsubstituted or substituted,
or a salt, enantiomer or zwitterion of the compound.

63. A method of reducing tissue damage due to an acute trauma in a subject, comprising administering to the subject a therapeutically effective amount of a protein phosphatase 2A (PP2A) inhibitor having the structure: 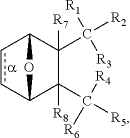
wherein
bond α is present or absent;
R₁ and R₂ is each independently H, O⁻ or OR₉,
where R₉ is H, alkyl, alkenyl, alkynyl or aryl,
or R₁ and R₂ together are ═O;
R₃ and R₄ are each different, and each is OH, O⁻, OR₉, O(CH₂)₁₋₆R₉, SH, S⁻, SR₉, 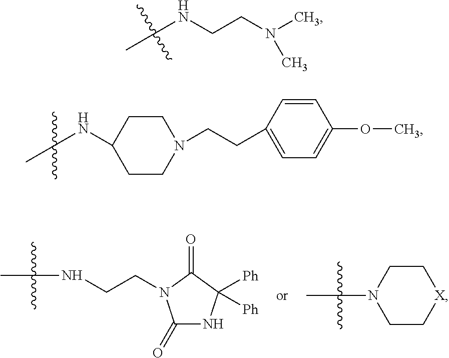
where X is O, S, NR₁₀, or N⁺R₁₀R₁₀,
where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O, 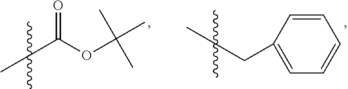
—CH₂CN, —CH₂CO₂R₁₁, —CH₂COR₁₁, —NHR₁₁ or —NH⁺(R₁₁)₂,
wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
where R₁₂ is H, alkyl, alkenyl, alkynyl or aryl; and
each occurrence of alkyl, alkenyl, or alkynyl is branched or unbranched, unsubstituted or substituted,
or a salt, enantiomer or zwitterion of the compound,
so as to thereby reduce tissue damage due to the acute trauma in the subject.

64. A method of reducing vascular leakage due to an acute trauma in a subject, comprising administering to the subject a therapeutically effective amount of a protein phosphatase 2A (PP2A) inhibitor having the structure: 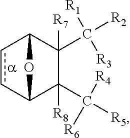
wherein
bond α is present or absent;
R₁ and R₂ is each independently H, O⁻ or OR₉,
where R₉ is H, alkyl, alkenyl, alkynyl or aryl, or R₁ and R₂ together are ═O;
R₃ and R₄ are each different, and each is OH, O⁻, OR₉, O(CH₂)₁₋₆R₉, SH, S⁻, SR₉, 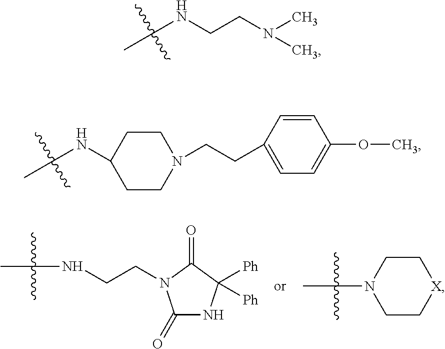
where X is O, S, NR₁₀, or N⁺R₁₀R₁₀,
where each R₁₀ is independently H, alkyl, C₂-C₁₂ alkyl, alkenyl, C₄-C₁₂ alkenyl, alkynyl, aryl, substituted aryl where the substituent is other than chloro when R₁ and R₂ are ═O, 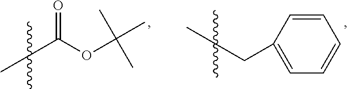
—CH₂CN, —CH₂CO₂R₁₁, —CH₂COR₁₁, —NHR₁₁ or —NH⁺(R₁₁)₂,
wherein each R₁₁ is independently alkyl, alkenyl or alkynyl, or H;
R₅ and R₆ is each independently H, OH, or R₅ and R₆ taken together are ═O;
R₇ and R₈ is each independently H, F, Cl, Br, SO₂Ph, CO₂CH₃, or SR₁₂,
where R₁₂ is H, alkyl, alkenyl, alkynyl or aryl; and
each occurrence of alkyl, alkenyl, or alkynyl is branched or unbranched, unsubstituted or substituted,
or a salt, enantiomer or zwitterion of the compound,
so as to thereby reduce vascular leakage due to the acute trauma in the subject.

65. (canceled)

66. The method of claim 63, wherein the acute trauma is due to surgical injury.

67. (canceled)
